(19) United States
(12) Patent Application Publication
(10) Pub. No.: US 2004/0131633 A1
(43) Pub. Date: Jul. 8, 2004

(54) Parasite antigens

(75) Inventors: John Timothy Ellis, Hornsby New South Wales (AU); Robert Atkinson, Irvinebank (AU); Cheryl Ryce, New South Wales (AU); Helen Elizabeth Quinn, Chapel Hill (AU); Catherine Margaret Miller, Roseville (AU); David Andrew Morrison, Uppsala (SE)

Correspondence Address: NIXON & VANDERHYE, PC, 1100 N GLEBE ROAD, 8TH FLOOR, ARLINGTON, VA, 22201-4714, US

(73) Assignee: University of Technology, Sydney

(21) Appl. No.: 10/608,436

(22) Filed: Jun. 30, 2003

Related U.S. Application Data
(63) Continuation-in-part of application No. 09/959,246, filed on Jan. 10, 2002.
Application No. 09/959,246 is a 371 of international application No. PCT/AU00/00354, filed on Apr. 20, 2000.

(30) Foreign Application Priority Data
Apr. 21, 1999 (AU) PP 9928
Apr. 20, 2000 (WO) PCT/AU00/00354
May 26, 1999 (WO) PCT/AU99/00405

(51) Int. Cl. A61K 39/02; C07K 14/195

(52) U.S. Cl. 424/190.1; 530/350

(57) ABSTRACT

The present invention relates to polypeptides from N. caninum which are capable of raising an immune response when administered to an animal. Such polypeptides can be used in vaccination strategies for protecting animals, such as cows and dogs, from neosporosis.

### TECHNICAL FIELD

The present invention is directed to parasite antigens, particularly Neospora antigens and uses thereof.

### BACKGROUND ART

Neospora caninum was first described in 1988 during a retrospective study of dogs previously diagnosed with fatal toxoplasmosis (Dubey et al. 1988). Since then, N. caninum has been shown to be one of the main causes of abortion in livestock around the world including, for example, the United States of America, Europe, New Zealand and Australia.

The genus Neospora was established in the family Sarcocystidae of the phylum Apicomplexa because of the close similarity in morphology between N. caninum and other cyst-forming coccidia such as Toxoplasma gondii. The complete life cycle of N. caninum is not known but it involves dogs as the definitive host (McAllister et al. 1998) and congenital infection has been recorded in dogs, cats, sheep, cattle, goats and horses.

The major clinical signs of neosporosis in congenitally infected pups is hindlimb paralysis which may rapidly progress to tetraplegia and death. Other symptoms include difficulty in swallowing, jaw paralysis, muscle flaccidity and atrophy. The disease does not usually become apparent until 3-6 weeks of age when limping or reduced limb movement may become apparent. Neosporosis occasionally manifests itself in older dogs, but congenital infection is more common. Transmission of infection to multiple, successive litters is possible. Histologically, necrotising nonsuppurative myositis of skeletal muscles and meningoencephalitis are the most consistent findings associated with canine neosporosis. Myositis is characterised by muscle atrophy, hypertrophy, necrosis and mononuclear cell infiltration. Mineralisation of muscle and acute myocarditis have also been reported. Parasites are most numerous in the central nervous system (CNS) and may be associated with lesions.

Vaccines for the control of neosporosis are not available, although infections in dogs (if caught early enough) may be treated with clindamycin. Therapy is not considered practical for cattle herds, and a vaccine is believed to represent one potential form of control. No information is currently available regarding the spread of the parasite, except through vertical transmission from mother to fetus. Infected cattle typically show no clinical signs of disease, although dams are considered at risk of suffering fetal loss or abortion during pregnancy. Therefore a vaccine that eliminates or reduces congenital infection, foetal loss and abortion, which are the main signs of neosporosis, in cattle and other livestock is considered essential.

There is a need for vaccines to raise a protective immune response in animals that are susceptible to neosporosis.

### DISCLOSURE OF THE INVENTION

The present inventors have identified a number of polypeptides from N. caninum that can be used to raise an immune response in animals that are susceptible to neosporosis.

Accordingly, in a first aspect the present invention provides a substantially purified polypeptide comprising a sequence selected from the group consisting of

a) a sequence provided in SEQ ID NO: 1;

b) a sequence which is at least 75% identical to (a);

c) a sequence provided in SEQ ID NO:4;

d) a sequence which is at least 75% identical to (c);

e) a sequence provided in SEQ ID NO:5;

f) a sequence which is at least 75% identical to (e);

g) a sequence provided in SEQ ID NO:3;

h) a sequence which is at least 60% identical to (g), and

i) an immunogenic fragment of any one of a) to h),

wherein the polypeptide, or fragment thereof, raises an immune response against N. caninum when administered to an animal.

Preferably, the polypeptide is at least 80% identical, more preferably at least 85% identical, more preferably at least 90% identical, more preferably at least 95% identical, and even more preferably at least 99% identical to any one of SEQ ID NO's 1 or 3 to 5.

Preferably, the polypeptide can be purified from N. caninum.

In one embodiment, the polypeptide comprises a sequence provided in SEQ ID NO:4.

In another aspect, the present invention provides a fusion protein comprising a polypeptide according to the invention fused to at least one heterologous polypeptide sequence.

Preferably, the at least one heterologous polypeptide sequence is selected from the group consisting of a polypeptide that enhances the stability of a polypeptide of the present invention, a polypeptide that enhances the immunogenicity of a polypeptide of the present invention, and a polypeptide that assists in purification of the fusion protein.

In a further aspect, the present invention provides a composition comprising a polypeptide according to the invention and a pharmaceutically acceptable carrier. Such compositions can be administered to an animal to raise an immune response to N. caninum.

It is preferred that the composition further comprises an adjuvant. Preferably, the adjuvant is selected from the group consisting of aluminum salts, water-in-soil emulsions, oil-in-water emulsions, saponin, QuilA and derivatives, iscoms, liposomes, cytokines including gamma interferon or interleukin 12, DNA, microencapsulation in a solid or semi-solid particle, Freunds complete and incomplete adjuvant or active ingredients thereof including muramyl dipeptide and analogues, DEAE dextran/mineral oil, Alhydrogel, Auspharm adjuvant, and Algammulin.

The present inventors have surprisingly found that a mixture of polypeptides of the invention provides an enhanced immune response when compared to use of the polypeptides as a homogenous population of antigens. Thus, in one preferred embodiment the composition comprises at least two polypeptides of the invention. In a particularly preferred embodiment, the composition comprises

a) a polypeptide comprising a sequence provided in SEQ ID NO:1, or a polypeptide which is at least 75% identical thereto, or an immunogenic fragment thereof, and

b) a polypeptide comprising a sequence provided in SEQ ID NO:2, or a polypeptide which is at least 75% identical thereto, or an immunogenic fragment thereof.

In a further aspect, the present invention provides a method for raising an immune response against N. caninum in an animal, the method comprising administering to the animal at least one composition according to the present invention.

The composition of the invention may be administered by any suitable means including, but not limited to, by injection via intramuscular, subcutaneous, intradermal or intraperitoneal routes or included as an additive in feed or water.

In yet another aspect, the present invention provides a method of treating or preventing an N. caninum infection in an animal, the method comprising administering to the animal at least one composition according to the present invention.

In a further aspect, the present invention provides for the use of a composition according to the present invention in the manufacture of a medicament for raising an immune response against N. caninum in an animal.

Preferably, the animal is a mammal. In one embodiment, the mammal is selected from the group consisting of, cows, horses, deer, sheep, goats and dogs.

In yet another aspect, the present invention provides an isolated polynucleotide, the polynucleotide having a sequence selected from:

a) a sequence of nucleotides shown in SEQ ID NO:7;

b) a sequence of nucleotides shown in SEQ ID NO:8;

c) a sequence of nucleotides shown in SEQ ID NO:9;

d) a sequence encoding a polypeptide, or immunogenic fragment thereof, or fusion protein according to the present invention;

e) a sequence capable of selectively hybridizing to any one of a) to d) under high stringency conditions; and

f) a sequence of nucleotides which is at least 70% identical to any one of a) to

wherein the polynucleotide encodes a polypeptide which raises an immune response against N. caninum when administered to an animal.

Preferably, the polynucleotide is at least 80% identical, more preferably at least 85% identical, more preferably at least 90% identical, more preferably at least 95% identical, and even more preferably at least 99% identical to any one of a) to c).

In yet another aspect, the present invention provides a polynucleotide obtainable by performing a nucleic acid amplification method on a N. caninum cDNA library with primers P20-ATG2F (5′ACGTATGGATCCGGCTTTGTCTACGATGAAC3′) SEQ ID NO: 14) and P20-pTrcR (5′ACGCATGAATTCTGTTTCTGAGTTCCCGCT3′) (SEQ ID NO: 15), or a fragment of said polynucleotide encoding a polypeptide which raises an immune response against N. caninum when administered to an animal.

Any nucleic acid amplification method can be used that is known in the art, for instance those techniques based on the polymerase chain reaction.

In a further aspect, the present invention provides a substantially purified polypeptide encoded by a polynucleotide of the invention, wherein the polypeptide raises an immune response against N. caninum when administered to an animal.

In a further aspect, the present invention provides a vector comprising at least one polynucleotide of the invention. The vectors may be, for example, plasmid, virus or phage vectors provided with an origin of replication, and preferably a promotor for the expression of the polynucleotide and optionally a regulator of the promotor. The vector may contain one or more selectable markers, for example an ampicillin resistance gene in the case of a bacterial plasmid or a neomycin resistance gene for a mammalian expression vector. The vector may be used in vitro, for example for the production of RNA or used to transfect or transform a host cell.

In one embodiment, the vector is a viral vector.

In another embodiment, the vector is a plasmid, preferably being VR1012, pTrcHisB or pET25b. It will be appreciated, however, that any other suitable plasmid could be used.

In another aspect, the present invention provides a host cell comprising a vector of the invention.

Preferably, the host cell is mammalian cell.

As is known in the art, an immune response can be provided through the use of DNA vaccines. Accordingly, in another aspect the present invention provides a DNA vaccine comprising at least one polynucleotide of the invention.

In another preferred embodiment, the polynucleotide is contained in a vector. More preferably, the vector is a viral vector.

In a further aspect, the present invention provides a method for raising an immune response against N. caninum in an animal, the method comprising administering to the animal a DNA vaccine of the invention.

In yet another aspect, the present invention provides a method of treating or preventing an N. caninum infection in an animal, the method comprising administering to the animal a DNA vaccine of the invention.

In a further aspect, the present invention provides for the use of a DNA vaccine according to the present invention in the manufacture of a medicament for raising an immune response against N. caninum in an animal.

It is also known in the art that an immune response can be provided by the consumption of a transgenic plant expressing an antigen. Thus, in a further aspect the present invention provides a transgenic plant which produces at least one polypeptide of the invention.

In yet another aspect, the present invention provides a method for raising an immune response against an N. caninum in an animal, the method comprising orally administering to the animal at least one transgenic plant of the invention.

In another aspect, the present invention provides a method of treating or preventing an N. caninum infection in an animal, the method comprising orally administering to the animal at least one transgenic plant of the invention.

In a further aspect, the present invention consists in the use of one or more of the polypeptides of the present invention in methods for detecting antibodies reactive or specific to Neospora. One particularly suitable use is a recombinant ELISA assay where detection of antibodies in a serum or blood sample from an animal that bind to one or more of the polypeptides would be indicative of the exposure to and/or infection of that animal with Neospora. Screening of animal herds for the presence of an immune response to Neospora can be carried out using the polypeptides according to the present invention in suitable immunological assays known to the art. Such tests would also be useful to determine whether immunisation with a composition of the present invention of an animal was successful at raising antibodies to Neospora.

Furthermore, antibodies raised against the polypeptides according to the present invention are suitable for use in assays to identify or diagnose the presence of Neospora. The antibodies can be raised in animals, for example laboratory animals, and purified for use by standard techniques. Similarly, monoclonal antibodies can also be produced in the usual manner from rodents immunised with a polypeptide so as to produce antibodies specific to Neospora.

In another aspect, the present invention provides an antibody, or fragment thereof raised against a polypeptide of the invention.

In a further aspect, the present invention provides a method of treating or preventing an N. caninum infection in an animal, the method comprising administering to the animal at least one antibody, or fragment thereof, according to the invention.

In another aspect, the present invention provides a substantially purified polypeptide which specifically binds to an antibody, or fragment thereof, according to the invention.

In a further aspect, the present invention provides a process for preparing a polypeptide according to the invention, the process comprising cultivating a host cell according to the invention under conditions which allow expression of the polynucleotide encoding the polypeptide, and recovering the expressed polypeptide. This process can be used for the production of commercially useful quantities of the encoded polypeptide.

In another aspect, the present invention provides a polypeptide produced by a process of the invention.

In yet another aspect, the present invention provides an oligonucleotide probe or primer, the probe or primer having a nucleotide sequence that hybridises selectively to a polynucleotide molecule of the present invention.

In a preferred embodiment, the oligonucleotide probe or primer includes at least 15 nucleotides, more preferably at least 18 nucleotides and more preferably at least 25 nucleotides.

In a further preferred embodiment, the oligonucleotide probe or primer is used as a detectable probe where the oligonucleotide is conjugated with a label such as a radioisotope, an enzyme, biotin, a fluorescent molecule or a chemiluminescent molecule.

Furthermore, the present invention also provides polynucleotides, oligonucleotides or antibodies of the invention in a composition with a suitable carrier or diluent.

As will be apparent, preferred features and characteristics of one aspect of the invention are applicable to many other aspects of the invention.

The invention is hereinafter described by way of the following non-limiting Examples and with reference to the accompanying figures.

### BRIEF DESCRIPTION OF THE DRAWINGS

FIG. 1. A) Gene organisation of NCGRA2 (SEQ ID NO: 12). Exons 1 and 2 are in bold and separated by a single intron. N represents an unidentified base. The start codon is at the beginning of exon 1; the stop codon at the end of exon 2. B) Open reading frame of NCGRA2 (SEQ ID NO: 9). C) Predicted amino acid sequence inferred from the open reading frame of GRA2 (SEQ ID NO: 3).

FIG. 2. Comparison of the amino acid sequence of Gra2 between N. caninum (NC) (SEQ ID NO: 3) and T. gondii (TG) (SEQ ID NO: 13). The unique C-terminal domain of NcGra2 is not shown.

FIG. 3. DNA vaccination of balb/c mice with either VR1012 (vector), pRevGRA2 or pGRA2 via the ear pinna. Graph shows change in mean body weight (MBW in g) with time (days post infection with N. caninum tachyzoites, dpi). The control was injected with endotoxin-free TE. The dashed line represents the change in weight of an unimmunised, uninfected group of mice. Numbers embedded in the graph represent the number of mice surviving at that time point.

FIG. 4. DNA vaccination of balb/c mice with either VR1012 (vector), pRevGRA2 or pGRA2 via the footpad. Graph shows change in mean body weight (MBW in g) with time (days post infection with N. caninum tachyzoites, dpi). The control was injected with endotoxin-free TE. The dashed line represents the change in weight of an unimmunised, uninfected group of mice. Numbers embedded in the graph represent the number of mice surviving at that time point.

FIG. 5. DNA vaccination of balb/c mice with either VRO112 (vector), pRevGRA2 or pGRA2 via the leg. Graph shows change in mean body weight (MBW in g) with time (days post infection with N. caninum tachyzoites; dpi). The control was injected with endotoxin-free TE. The dashed line represents the change in weight of an unimmunised, uninfected group of mice. Numbers embedded in the graph represent the number of mice surviving at that time point.

FIG. 6. ELISA performed using recombinant (his-tagged) NcGra2 with sera from experimentally infected mice. The experimental groups were: Cnp; control group of non-pregnant mice, 1) non-pregnant mice infected with 10⁶ tachyzoites of N. caninum (NC-Liverpool) Cp, control group of un-infected, pregnant mice, 2) pregnant mice infected with 10⁷ tachyzoites of N. caninum (NC-Liverpool); 3) pregnant mice infected with 10⁶ tachyzoites of N. caninum (NC-SweB1).

FIG. 7: 24B cDNA sequence (SEQ ID NO 10), region in bold is the ORF (SEQ ID NO: 7).

FIG. 8. A) 24B amino acid sequence (SEQ ID NO: 1). B) C-terminal fragment of 24B (SEQ ID NO: 4).

FIG. 9. Genomic DNA sequence of the 24B gene (SEQ ID NO: 11). The three regions making up the OFF are in bold and underlined.

FIG. 10. NcP20 coding sequence (SEQ ID NO: 8).

FIG. 11. A) NcP20 amino acid sequence (SEQ ID NO:2). B) N-terminal fragment of NcP20 (SEQ ID NO: 5). C) NcP20 fragment (SEQ ID NO: 6).

FIG. 12. Shows the results of a vaccination trial using recombinant NcP20 fragment (SEQ ID NO: 6). The graph shows a plot of mean group body weight (MGW) against days post infection (DPI) with N. caninum.

FIG. 13. Shows ELISA optical density (absorbance) reading for three independent test sera of nice for the presence of antibodies to NcP20.

Key to the Sequence Listing

SEQ ID NO: 1—N. caninum 24B protein.

SEQ ID NO: 2—N. caninum NcP20 protein.

SEQ ID NO: 3—N. caninum Gra2 protein.

SEQ ID NO: 4—C-terminal fragment of N. caninum 24B protein.

SEQ ID NO: 5—N-terminal fragment of N. caninum NcP20 protein.

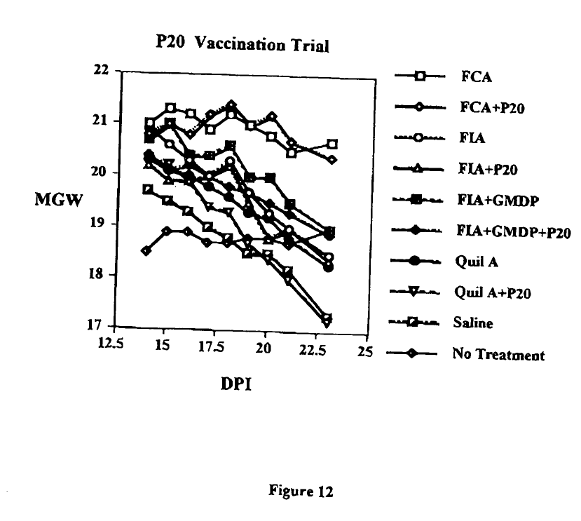

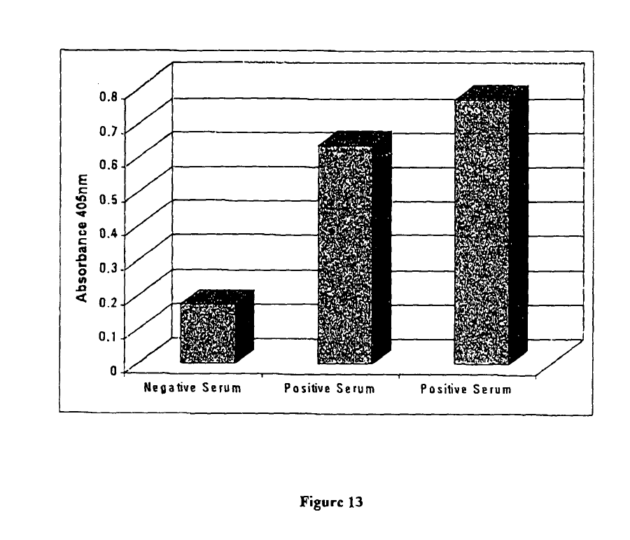

SEQ ID NO: 6—Fragment of N. caninum NcP20 used in vaccination and ELISA experiments (see FIGS. 12 and 13).

SEQ ID NO: 7—ORF encoding N. caninum 24B.

SEQ ID NO: 8—ORF encoding N. caninum NcP20.

SEQ ID NO: 9—ORF encoding N. caninum Gra2.

SEQ ID NO: 10—Complete N. caninum 24B cDNA sequence, including 5′ and 3‘UTR’s.

SEQ ID NO: 11—Sequence of N. caninum 24B gene.

SEQ ID NO: 12—Sequence of N. caninum Gra2 gene.

SEQ ID NO: 13—Partial sequence of T. gondii Gra2 protein.

SEQ ID NO's: 14 to 32 and 34 to 59—PCR primers.

SEQ ID NO: 33: Gra2 signal sequence.

### MODES OF CARRYING OUT THE INVENTION

Definitions

By “treating or preventing an N. caninum infection in an animal” we mean the reduction or prevention of at least one symptom associated with the infection.

An “immune response” is the total immunological reaction of an animal to an immunogenic stimulus. In general there are considered to be two types of immune responses produced by two phenotypically different populations of lymphocytes. B cells are responsible for humoral immunity, producing antibodies that circulate in the blood stream, whereas T cells are responsible for cell-mediated immunity. As used herein, an immune response can be any or all of the immune systems reaction to being exposed to a polypeptide of the invention.

Upon exposure to a polypeptide of the invention, an animals immune system is stimulated to produce an immune response which will recognise N. caninum. N. caninum infection, leading to the disease of neosporosis, has a number of manifestations depending on the animal infection. For instance, in cattle infection can result in fetal death and/or abortion. In dogs, N. caninum invades the central nervous system leading to inflammation and paralysis of the hind limbs. Accordingly, the immune response induced by the methods of the present invention can result in a reduction in the parasitaemia of N. caninum within an animal, and/or increase the ability of the animal to resist N. caninum infection, and/or alleviate at least one symptom of N. caninum infection such as central nervous system-related disease or fetal loss, which results from transplacental transmission of the parasite during pregnancy.

An “immunogen” or an “antigen” is a molecule that when administered into an animal causes an immune response.

An “immunogenic fragment” or “antigenic fragment” is a portion of a polypeptide of the invention that raises an immune response against N. caninum when administered to an animal. Such fragments are typically at least 6 amino acids in length.

As used herein, the term “effective amount” means a sufficient quantity of the polypeptide to produce an immune response upon administration of the polypeptide to an animal.

By “isolated polynucleotide” we mean a polynucleotide which have generally been separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid molecule”.

By “substantially purified polypeptide” we mean a polypeptide that has generally been separated from the lipids, nucleic acids, other polypeptides, and other contaminating molecules with which it is associated in its native state. Preferably, the substantially purified polypeptide is at least 60% free, preferably at least 75% free, and most preferably at least 90% free from other components with which they are naturally associated.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

General Methods

Unless specifically defined otherwise, all technical and scientific terms used herein shall be taken to have the same meaning as commonly understood by one of ordinary skill in the art (e.g., in cell culture, molecular genetics, immunology, nucleic acid chemistry, hybridisation techniques and biochemistry).

Unless otherwise indicated, the recombinant DNA and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Harnes (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

Polynucleotides

The polynucleotide encoding a polypeptide of the invention may be obtained from any cDNA library prepared from tissue or organisms believed to express the gene mRNA and to express it at a detectable level. The gene sequences can also be obtained from a genomic library or genomic DNA.

Libraries are screened with probes or analytical tools designed to identify the gene of interest or the protein encoded by it. For cDNA expression libraries, suitable probes include monoclonal or polyclonal antibodies that recognise and specifically bind the protein; oligonucleotides of about 20-80 bases in length that encode known or suspected portions of cDNA from the same or different species, and/or complementary or homologous cDNAs or fragments thereof that encode the same or a hybridising gene. Appropriate probes for screening genomic DNA libraries include, but are not limited to, oligonucleotides; cDNAs or fragments thereof that encode the same or hybridising DNA including expressed sequence tags and the like; and/or homologous genomic DNAs or fragments thereof. Screening the cDNA or genomic library with the selected probe may be conducted using standard procedures as described in chapters 10-12 of Sambrook et al (supra).

An alternative means to isolate a gene encoding is to use polymerase chain reaction (PCR) methodology as described in section 14 of Sambrook et al (supra). This method requires the use of oligonucleotide primers that will hybridise to the gene.

The oligonucleotide sequences selected as primers should be of sufficient length and sufficiently unambiguous that false positives are minimised. The actual nucleotide sequence(s) is usually based on conserved or highly homologous nucleotide sequences or regions of the gene. The oligonucleotides may be degenerate at one or more positions. The use of degenerate oligonucleotides may be of particular importance where a library is screened from a species in which preferential codon usage in that species is known. The oligonucleotide must be labelled such that it can be detected upon hybridisation to DNA in the library being screened. The preferred method of labelling is to use ³²P-tabelled ATP with polynucleotide kinase, as is well known in the art, to radiolabel the oligonucleotide. However, other methods may be used to label the oligonucleotide, including, but not limited to, biotinylation or enzyme labelling.

Nucleic acid having all the protein coding sequence is obtained by screening selected cDNA or genomic libraries, and if necessary, using conventional primer extension procedures as described in section 7.79 of Sambrook et al. (supra), to detect precursors and processing intermediates of mRNA that may not have been reverse-transcribed into cDNA.

Another alternative method for obtaining the gene of interest is for it to be chemically synthesized. These methods include triester, phosphite, phosphoramidite and H-Phosphonate methods, PCR and other autoprimer methods, and oligonucleotide syntheses on solid supports. These methods may be used if the entire nucleic acid sequence of the gene is known, or the sequence of the nucleic acid complementary to the coding strand is available, or alternatively if the target amino acid sequence is known, one may infer potential nucleic acid sequences using known and preferred coding residues for each amino acid residue.

Mutants, Variants and Homology—Nucleic Acids

Mutant polynucleotides will possess one or more mutations which are deletions, insertions, or substitutions of nucleotide residues. Mutants can be either naturally occurring (that is to say, isolated from a natural source) or synthetic (for example, by performing site-directed mutagensis on the DNA). It is thus apparent that polynucleotides of the invention can be either naturally occurring or recombinant (that is to say prepared using recombinant DNA techniques). Mutant polynucleotides of the invention can be prepared, and the immunogenicity of the polypeptides they encode be tested, using techniques known in the art.

The % identity of a polynucleotide is determined by GAP (Needleman and Wunsch, 1970) analysis (GCG program) with a gap creation penalty=5, and a gap extension penalty=0.3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. Even more preferably, the query sequence is at least 300 nucleotides in length and the GAP analysis aligns the two sequences over a region of at least 300 nucleotides.

When used herein, stringent conditions or “high stringency conditions” are those that (1) employ low ionic strength and high temperature for washing, for example, 0.015 M NaCl/0.0015 M sodium citrate/0/1% NaDodSO₄ at 65° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

An allelic variant will be a variant that is naturally occurring within an individual organism.

Polypeptide sequences are “homologous” or “species homologues” if they are related by divergence from a common ancestor. Consequently, a species homologue of a polypeptide will be the equivalent polypeptide which occurs naturally in another species or strains of a species. Within any one species a homologue may exist as numerous allelic variants, and these will be considered homologues of the polypeptide. Allelic variants and species homologues can be obtained by following standard techniques known to those skilled in the art. Preferred species homologues include those obtained from representatives of the same Order, more preferably the same Family and even more preferably the same Genus.

Mutants, Variants and Homolog—Proteins

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids.

Mutant polypeptides will possess one or more mutations which are deletions, insertions, or substitutions of amino acid residues. Mutants can be either naturally occurring (that is to say, purified or isolated from a natural source) or synthetic (for example, by performing site-directed mutagensis on the encoding DNA). It is thus apparent that polypeptides of the invention can be either naturally occurring or recombinant (that is to say prepared using recombinant DNA techniques). Mutant polypeptides of the invention can be prepared, and their immunogenicity tested, using techniques known in the art.

Amino acid sequence variants can be prepared by introducing appropriate nucleotide changes into DNA, or by in vitro synthesis of the desired polypeptide. Such variants include, for example, deletions, insertions or substitutions of residues within the amino acid sequence. A combination of deletion, insertion and substitution can be made to arrive at the final construct, provided that the final protein product possesses the desired characteristics. The amino acid changes also may alter post-translational processes such as changing the number or position of glycosylation sites, altering the membrane anchoring characteristics, altering the intra-cellular location by inserting, deleting or otherwise affecting the transmembrane sequence of the native protein, or modifying its susceptibility to proteolytic cleavage.

In designing amino acid sequence variants, the location of the mutation site and the nature of the mutation will depend on characteristic(s) to be modified. The sites for mutation can be modified individually or in series, eg., by (1) substituting first with conservative amino acid choices and then with more radical selections depending upon the results achieved, (2) deleting the target residue, or (3) inserting residues of other ligands adjacent to the located site.

A useful method for identification of residues or regions for mutagenesis is called “alanine scanning mutagenesis” as described by (Cunningham and Wells, 1989). Here, a residue or group of target residues are identified (eg., charged residues such as Arg, Asp, His, Lys, and Glu) and replaced by a neutral or negatively charged amino acid (most preferably alanine or polyalanine) to affect the interaction of the amino acids with the surrounding aqueous environment in or outside the cell. Those domains demonstrating functional sensitivity to the substitutions then are refined by introducing further or other variants. Thus, while the site for introducing an amino acid sequence variation is predetermined, the nature of the mutation per se need not be predetermined. For example, to optimise the performance of a mutation at a given site, alanine scanning or random mutagenesis may be conducted at the target codon or region and the expressed variants are screened for the optimal combination of desired activity.

There are two principal variables in the construction of amino acid sequence variants: the location of the mutation site and the nature of the mutation. These may represent naturally occurring alleles or predetermined mutant forms made by mutating the DNA either to arrive at an allele or a variant not found in nature. In general, the location and nature of the mutation chosen will depend upon the characteristic to be modified.

Amino acid sequence deletions generally range from about 1 to 30 residues, more preferably about 1 to 10 residues and typically about 1 to 5 contiguous residues.

Amino acid sequence insertions include amino and/or carboxyl-terminal fusions ranging in length from one residue to polypeptides containing a hundred or more residues, as well as intrasequence insertions of single or multiple amino acid residues. Other insertional variants include the fusion of the N- or C-terminus of the proteins to an immunogenic polypeptide eg. bacterial polypeptides such as betalactamase or an enzyme encoded by the E. coli trp locus, or yeast protein, bovine serum albumin, and chemotactic polypeptides. C-terminal fusions with proteins having a long half-life such as immunoglobulin constant regions (or other immunoglobulin regions), albumin, or ferritin, are included.

Another group of variants are amino acid substitution variants. These variants have at least one amino acid residue in the protein molecule removed and a different residue inserted in its place. The sites of greatest interest for substitutional mutagenesis include sites identified as the active site(s). Other sites of interest are those in which particular residues obtained from various species are identical. These positions may be important for biological activity. These sites, especially those falling within a sequence of at least three other identically conserved sites, are substituted in a relatively conservative manner. Such conservative substitutions are shown in Table 1 under the heading of “preferred substitutions”. If such substitutions result in a change in biological activity, then more substantial changes, denominated exemplary substitutions in Table 1, or as further described below in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydro-phobicity of the molecule at the target site, or (c) the bulk of the side chain. Naturally occurring residues are divided into groups based on common side chain properties:

(1) hydrophobic: norleucine, met, ala, val, leu, ile;

(2) neutral hydrophilic; cys, ser, thr;

(3) acidic: asp, glu;

(4) basic: asn, gin, his, lys, arg;

(5) residues that influence chain orientation: gly, pro, and

(6) aromatic: trp, tyr, phe

TABLE 1
Preferred amino acid substitutions

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro | pro |
| His (H) | asn, gln; lys; arg | arg |
| Ile (I) | leu; val; met; ala; phe | leu |
| | norleucine | |
| Leu (L) | norleucine, ile, val; | ile |
| | met; ala; phe | |
| Lys (K) | arg, gln; asn | arg |
| Met (M) | leu; phe, ile; | leu |
| Phe (F) | leu; val, ile; ala | leu |
| Pro (P) | gly | gly |
| Ser (S) | thr | thr |
| Thr (T | ser | ser |
| Trp (W) | tyr | tyr |
| Tyr (Y) | trp; phe; thr; ser | phe |
| Val (V) | ile; leu; met; phe | leu |
| | ala; norleucine | |

Non-conservative substitutions will entail exchanging a member of one of these classes for another. It is generally preferred that encoded peptides differing from the determined polypeptide contain substituted codons for amino acids which are from the same group as that of the amino acid replaced. Thus, in general, the basic amino acids Lys, Arg, and His are interchangeable; the acidic amino acids Asp and Glu are interchangeable; the neutral polar amino acids Ser, Thr, Cys, Gln, and Asn are interchangeable; the nonpolar aliphatic amino acids Gly, Ala, Val, Ile, and Leu are conservative with respect to each other (but because of size, Gly and Ala are more closely related and Val, Ile and Leu are more closely related), and the aromatic amino acids Phe, Trp and Tyr are interchangeable.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Also included within the scope of the invention are biologically active fragments of the polypeptides of the present invention. By “biologically active fragment” we mean a fragment of a sequence of the present invention which retains at least one of the activities of the native polypeptide.

Most preferably, a “biologically active fragment” of the present invention is capable of raising an immune response against N. caninum when the fragment is administered to an animal. Such fragments are also referred to herein as an “immunogenic fragment” or an “antigenic fragment”.

As would be known to the skilled addressee, techniques for identifying a biologically active fragment or mutant of a polypeptide of the present invention which is capable of raising an immune response against N. caninum in an animal are well known in the art. For instance, substitutions and/or deletions can be made to the polypeptides of the present invention and the resulting fragment/mutant tested for its ability to raise an immune response against N. caninum.

Polypeptides of the present invention can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Vectors

One embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA based vector that is capable of transforming a host cell and effecting expression of a specified nucleic acid molecule. Preferably, the expression vector is also capable of replicating within the host cell. Expression vectors can be either prokaryotic or eukaryotic, and are typically based on viruses and their genomes or bacterial plasmids. Expression vectors of the present invention include any vectors that function (i.e., direct gene expression) in recombinant cells of the present invention, including in bacterial, fungal, endoparasite, arthropod, animal, and plant cells. Preferred expression vectors of the present invention can direct gene expression in bacterial, yeast, protozoal, plant and mammalian cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, plant and mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins).

Recombinant molecules of the present invention may also (a) contain secretory signals (i.e., signal segment nucleic acid sequences) to enable an expressed polypeptide of the present invention to be secreted from the cell that produces the polypeptide and/or (b) contain fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments, as well as natural signal sequences. In addition, a nucleic acid molecule of the present invention can be joined to a fusion segment that directs the encoded protein to the proteosome, such as a ubiquitin fusion segment. Recombinant molecules may also include intervening and/or untranslated sequences surrounding and/or within the nucleic acid sequences of nucleic acid molecules of the present invention.

Host Cells

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Suitable host cells for cloning or expressing the protein(s) disclosed herein are the prokaryote, protozoan, yeast, or higher eukaryote cells. Suitable prokaryotes include eubacteria, such as Gram-negative or Gram-positive organisms, for example, Escherichia coli, Bacilli such as B. subtilis or B. thuringiensis, Pseudornoias species such as P. aeruginosa, Salmonella typhimurium or Serratia marcescens.

Eukaryotic microbes such as protozoans, filamentous fungi or yeast are suitable hosts for expressing the protein(s) of the present invention. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as e.g. K. lactis; filamentous fungi such as, e.g. Neurospora, or Penicillium and Aspergilliis hosts such as A. nidulans and A. niger. Other parasites, such as T. gondii, are also appropriate hosts for expressing the protein according to the present invention.

Suitable higher eukaryotic host cells can be cultured vertebrate, invertebrate or plant cells. Insect host cells from species such as Spodoptera frugiperda, Aedes aegti, Aedes albopictus, Drosophila melanogaster, and Bombyx mori can be used. Plant cell cultures of cotton, corn, potato, soybean, tomato, and tobacco can be utilised as hosts. Typically, plant cells are transfected by incubation with certain strains for the bacterium Agrobacterium tumefaciens.

Propagation of vertebrate cells in culture (tissue culture) has become a routine procedure in recent years. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK ATCC CCL 10); Chinese hamster ovary cells/-DHFR(CHO); mouse sertoli cells, monkey kidney cells (CV1 ATCC CCL 70); human cervical carcinoma cells (HELA, ATCC CCL 2); canine kidney cells MDCK ATCC CCL 34), and a human hepatoma cell line (Hep G2). Preferred host cells are canine kidney cells and Chinese hamster ovary cells.

Recombinant DNA technologies can be used to improve expression of transformed polynucleotide molecules by manipulating, for example, the number of copies of the polynucleotide molecules within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts. The activity of an expressed recombinant protein of the present invention may be improved by fragmenting, modifying, or derivatizing polynucleotide molecules encoding such a protein.

Protozoan parasites, such as the related species Toxoplasma gondii, are considered ideal vectors for the expression of N. caninum genes. The development of a wide range of molecular genetics tools for T. gondii, such as transformation, gene disruption technologies and expression vector systems has propelled this organism into the forefront of parasite genetics, and these technologies are now considered state of the art for this organism (see, for example, Knoll et al. 2001; Sibley et al. 2002). Some of these techniques have also been developed for N. caninum showing they are easily transferable to this species (Howe and Sibley, 1997).

Compositions and Vaccines

Vaccines may be prepared from one or more polypeptides of the invention. The preparation of vaccines which contain an immunogenic polypeptide(s) as active ingredient(s), is known to one skilled in the art. Typically, such vaccines are prepared as injectables, either as liquid solutions or suspensions, solid forms suitable for solution in, or suspension in, liquid prior to injection may also be prepared. The preparation may also be emulsified, or the protein encapsulated in liposomes. The active immunogenic ingredients are often mixed with carriers/excipients which are pharmaceutically acceptable and compatible with the active ingredient. Suitable carriers/excipients are, for example, water, saline, dextrose, glycerol, ethanol, or the like and combinations thereof.

In addition, if desired, the vaccine may contain minor amounts of auxiliary substances such as wetting or emulsifying agents, pH buffering agents, and/or adjuvants which enhance the effectiveness of the vaccine.

As used herein, the term “adjuvant” means a substance that non-specifically enhances an immune response to an immunogen. Examples of adjuvants which may be effective include but are not limited to aluminum hydroxide, Quil A, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBL which contains three components extracted from bacteria, monophosphoryl lipid A, trehalose dimycolate and cell wall skeleton (MPL(+)TDM(+)CWS) in a 2% squalene/Tween 80 emulsion. Further examples of adjuvants include aluminum phosphate, aluminum potassium sulfate (alum), bacterial endotoxin, lipid X, Corynebacterium parvum (Propionobacterium acnes), Bordetella pertussis, polyribonucleotides, sodium alginate, lanolin, lysolecithin, vitamin A, saponin, liposomes, levamisole, DEAF-dextran, blocked copolymers or other synthetic adjuvants. A preferred adjuvant is the CSIRO adjuvant (which contains 1 mg Quil A, 10 mg DEAE Dextran, 1.2 ml Montamide ISA 50V, and 0.8 ml PBS-per 2 ml). Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

The proportion of immunogen and adjuvant can be varied over a broad range so long as both are present in effective amounts. For example, aluminum hydroxide can be present in an amount of about 0.5% of the vaccine mixture (Al₂O₃ basis). Conveniently, the vaccines are formulated to contain a final concentration of immunogen in the range of from 0.2 to 200 μg/ml, preferably 5 to 50 μg/ml, most preferably about 15 μg/ml.

After formulation, the vaccine may be incorporated into a sterile container which is then sealed and stored at a low temperature, for example 4° C., or it may be freeze-dried. Lyophilisation permits long-term storage in a stabilised form.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

DNA Vaccines

DNA vaccination involves the direct in vivo introduction of DNA encoding an antigen into cells and/or tissues of an animal for expression of the antigen by the cells of the animal's tissue. Such vaccines are termed herein “DNA vaccines” or “nucleic acid-based vaccines.” Examples of DNA vaccines are described in U.S. Pat. No. 5,939,400, U.S. Pat. No. 6,110,898, WO 95/20660 and WO 93/19183. The ability of directly injected DNA that encodes an antigen to elicit a protective immune response has been demonstrated in numerous experimental systems (see, for example, Cardoso et al., 1996; Sedegah et al., 1994: Montgomery et al., 1993; Wang et al., 1993; Xiang et al., 1994, Yang et al., 1997).

To date, most DNA vaccines in mammalian systems have relied upon viral promoters derived from cytomegalovirus (CMV). These have had good efficiency in both muscle and skin inoculation in a number of mammalian species. A factor known to affect the immune response elicited by DNA immunization is the method of DNA delivery, for example, parenteral routes can yield low rates of gene transfer and produce considerable variability of gene expression (Montgomery et al., 1993). High-velocity inoculation of plasmids, using a gene-gun, enhanced the immune responses of mice (Fynan et al., 1993; Eisenbraun et al., 1993), presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

Vaccines Derived from Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Exemplary dicotyledons include corn, tomato, potato, bean, soybean, and the like. Typically the transgenic plant is routinely used as a feed source for farm animals, particularly cows.

Transgenic plants, as defined in the context of the present invention include plants (as well as parts and cells of said plants) and their progeny which have been genetically modified using recombinant DNA techniques to cause or enhance production of at least one polypeptide of the present invention in the desired plant or plant organ.

Several techniques exist for introducing foreign genetic material into a plant cell, and for obtaining plants that stably maintain and express the introduced gene. Such techniques include acceleration of genetic material coated onto microparticles directly into cells (see, for example, U.S. Pat. No. 4,945,050 and U.S. Pat. No. 5,141,131). Plants may be transformed using Agrobacterium technology (see, for example, U.S. Pat. No. 5,177,010, U.S. Pat. No. 5,104,310, U.S. Pat. No. 5,004,863, U.S. Pat. No. 5,159,135). Electroporation technology has also been used to transform plants (see, for example, WO 87/06614, U.S. Pat. Nos. 5,472,869, 5,384,253, WO 92/09696 and WO 93/21335). In addition to numerous technologies for transforming plants, the type of tissue which is contacted with the foreign genes may vary as well. Such tissue would include but would not be limited to embryogenic tissue, callus tissue type I and II, hypocotyl, meristem, and the like. Almost all plant tissues may be transformed during development and/or differentiation using appropriate techniques described herein.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, regulated, or cell- or tissue-specific expression), a transcription initiation start site, a ribosome binding site, an RNA processing signal, a transcription termination site, and/or a polyadenylation signal.

Examples of plant promoters include, but are not limited to ribulose-1,6-bisphosphate carboxylase small subunit, beta-conglycinin promoter, phaseolin promoter, ADH promoter, heat-shock promoters and tissue specific promoters. Promoters may also contain certain enhancer sequence elements that may improve the transcription efficiency. Typical enhancers include but are not limited to Adh-intron 1 and Adh-intron 6.

Constitutive promoters direct continuous gene expression in all cells types and at all times (e.g., actin, ubiquitin, CaMV 35S). Tissue specific promoters are responsible for gene expression in specific cell or tissue types, such as the leaves or seeds (e.g., zein, oleosin, napin, ACP, globulin and the like) and these promoters may also be used. Promoters may also be active during a certain stage of the plants' development as well as active in plant tissues and organs. Examples of such promoters include but are not limited to pollen-specific, embryo specific, corn silk specific, cotton fiber specific, root specific, seed endosperm specific promoters and the like.

Under certain circumstances it may be desirable to use an inducible promoter. An inducible promoter is responsible for expression of genes in response to a specific signal, such as: physical stimulus (heat shock genes); light (RUBP carboxylase); hormone (Em); metabolites; and stress. Other desirable transcription and translation elements that function in plants may be used.

In addition to plant promoters, promoters from a variety of sources can be used efficiently in plant cells to express foreign genes. For example, promoters of bacterial origin, such as the octopine synthase promoter, the nopaline synthase promoter, the mannopine synthase promoter; promoters of viral origin, such as the cauliflower mosaic virus (35S and 19S) and the like may be used.

A number of plant-derived edible vaccines are currently being developed for animal pathogens (Hood and Jilka, 1999). Immune responses have also resulted from oral immunization with transgenic plants producing virus-like particles (VLPs), or chimeric plant viruses displaying antigenic epitopes (Mason et al., 1996; Modelska et al., 1998; Kapustra et al., 1999; Brennan et al., 1999). It has been suggested that the particulate form of these VLPs or chimeric viruses may result in greater stability of the antigen in the stomach, effectively increasing the amount of antigen available for uptake in the gut (Mason et al. 1996, Modelska et al. 1998).

Antibodies

The invention also provides monoclonal or polyclonal antibodies to polypeptides of the invention, or antigenic fragments thereof. Thus, the present invention further provides a process for the production of monoclonal or polyclonal antibodies to polypeptides of the invention

If polyclonal antibodies are desired, a selected mammal (e.g., mouse, rabbit, goat, cow, etc.) is immunised with an immunogenic polypeptide of the present invention. Serum from the immunised animal is collected and treated according to known procedures. If serum containing polyclonal antibodies to a polypeptide of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention, or antigenic fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against polypeptides of the present invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)_{z} fragments, as well as single chain antibodies (scFv). Furthermore, the antibodies and fragments thereof may be humanised antibodies, for example as described in EP-A-239400.

Antibodies may be used in a method for detecting polypeptides of the invention present in biological samples by a method which comprises:

(a) providing an antibody of the invention;

(b) incubating a biological sample with said antibody under conditions which allow for the formation of an antibody-antigen complex; and

(c) determining whether an antibody-antigen complex comprising said antibody is formed

Antibodies of the invention may be bound to a solid support and/or packaged into kits in a suitable container along with suitable reagents, controls, instructions and the like

### EXAMPLE 1

### GRA-2

Materials and Methods

Parasite Culture

N. caninum isolates NC-Liverpool (Barber et al. 1995) and NC-SweB1 (Stenlund et al. 1997) were propagated in-vitro in Vero host cells according to established procedures (Barber et al. 1995).

Immnoscreening of Expression Libraries

In work leading up to the present invention, the inventors screened a recombinant cDNA expression library with sera obtained from an infected cow. Sera were obtained from cows in a herd where Neospora-associated abortion was common. The sera obtained from each cow was screened by western blotting using N. caninum tachyzoite antigen in order to identify diagnostic antigens. Serum from cow X (identified in this way) was prepared for immunoscreening by preabsorption against Escherichia coli and Don recombinant lambda ZAP bacteriophage by pseudoscreening in order to remove non-specific cross reactive antibodies from it prior to use (Sambrook et al., supra).

Total RNA was extracted from cell-cultured tachyzoites of NC-Liverpool. Briefly, tachyzoites were lysed and vortexed in a strong denaturing buffer containing 5.7M guanidium thiocyanate, 100 mM sodium acetate pH 5.2, 10 mM EDTA and 100 mM 2-mercaptoethanol. Insoluble debris was removed by centrifugation (10,000 g, 4° C., 10 min) and the supernatant was precipitated overnight, recentrifuged, resuspended and subjected to a phenolichoroform step in lysis buffer containing 4.5M guanidium thiocyanate. The aqueous phase was precipitated overnight, centrifuged and the pellet was washed twice and stored as a precipitate in 70% ethanol at −20° C. Messenger RNA was purified from total RNA using oligo-dT cellulose chromatography. RNA pellets were centrifuged (20 min, 10,000 g, 4° C.), pooled and resuspended in 5 ml of TS buffer (10 mM Tris, 0.1% SDS) for 15 min at 65° C. The solution was then cooled rapidly on ice and sodium chloride added to a final concentration of 400 mM. The solution was then passed through a sterile syringe containing oligo-dT cellulose (Clontech), eluate was collected in baked cuvettes and the A₂₆₀ of consecutive fractions was read on a spectrophotometer. After the major peak of poly-A⁻ was eluted of the bound poly-A⁽⁺⁾ (mRNA) was collected by flushing the column with TS buffer. Fractions containing the poly-A⁽⁺⁾ A₂₆₀ peaks were then precipitated and stored at −70° C. Reagents and equipment for cDNA library construction were supplied by Stratagene. The mRNA was centrifuged (20 min, 10,000 g, 4° C.) and resuspended in DEPC-treated water for 15 min at 65° C. The solution was cooled rapidly on ice and single stranded cDNA was synthesised in first stand buffer containing a poly-dT primer with an internal Xho1 restriction site. Second strand synthesis, blunt ending, addition of EcoR1 adaptors and Xho1 digestion were performed following instructions provided by the manufacturer. Double stranded cDNA was then size-fractionated on a Sephacryl S-500 column (Clontech) to remove short molecules. Prepared cDNA was ligated into EcoR1/Xho1 digested arms of the UNI-ZAP XR bacteriophage vector and packaged into viable phage using Gigapack Gold III packagene extracts. The titre of the cDNA library was determined by plating serially diluted aliquots onto E. coli. The primary library contained 1.1×10⁶ recombinant clones.

The cDNA library was screened with preabsorbed bovine anti-Areosora antisera from cow X using standard procedures. Briefly, filters containing plaques were incubated in Tris-buffered saline supplemented with 5% skim milk powder either overnight at 4° C., or for 1 hour at room temperature (RT) in order to prevent non-specific antibody binding. Filters were then incubated for 45 min at RT with either a negative bovine control serum (sourced from a Neospora-free herd of dairy cattle) or from cow X, diluted 1/50 or 1/100. Filters were then washed in Tris-buffered saline-Tween and further incubated for 45 rain at RT in a 1/500 dilution of anti-bovine IgG conjugated to alkaline phosphatase (Sigma). Washing was repeated and membranes were placed in the developing solution of nitroblue tetrozoleum and 5-bromo-4-chloro-3-indolyl-phosphate (Sigma) for 20 min at RT. Recombinant clones expressing N. caninum antigens were picked and rescreened until a pure population of phages was produced.

Characterisation of Cloned Sequences

The cloned DNAs coding for N. caninum specific antigens were characterised as follows. A recombiant phage plaque was picked into double distilled water and subjected to PCR amplication using primers FpB (5′GTAAAACGACGGCCAGT3′) (SEQ ID NO: 16) and RpB2 (5′GCCGCTCTAGAACTA3′) (SEQ ID NO: 17). A 50 μl PCR reaction was used with 2.5 mM MgCl₂, 200 μM dNTP, 25 pmol primer with cycling conditions, 1 cycle, 95° C., 3 min; 25 cycles, 95° C., 1 min, 52° C., 1 min, 72° C., 2.5 min and 1 cycle, 72° C., 5 min. Five μl of the PCR product was run on a 1% agarose gel to estimate size and amount of product obtained. The PCR product was then purified using a Qiagen column and sequenced by cycle sequencing and the aid of an ABI automated sequencer. The non-redundant nucleotide sequence database maintained by the National Center for Bioinformatics (NCBI) and the Apicomplexa nucleotide sequence database at the Parasite Genome Blast Server (PGBS; http://www.ebi.ac.uk/parasite/parasite_blastserver.html) (Gish, W., personal communication) were searched with the sequences obtained using the program BlastN in order to detect homologies with nucleotide sequences currently in the nucleotide sequence databases. The recombinants were then grouped according to their database matches. Further searches were also made of the Toxoplasma Database of Clustered ESTS (ToxoDB; http://www.cibil.upenn.edu/agi-bin/ParaDBs/Toxoplasma/index.html) (Kissinger et al., 2003).

In order to obtain the complete sequence of the N. caninum cDNAs isolated, a PCR product derived for each cloned insert was cloned into the plasmid vector pGEM-T and the inserts were sequenced by cycle sequencing and a LiCOR sequencer. The sequences obtained were compiled using Assembly Align.

Northern blotting

Total RNA was extracted from N. caninum tachyzoites using a Qiagen RNeasy Mini kit following the manufacturers instructions. The quality of the RNA was checked by agarose gel electrophoresis. For northern blotting, 5 μg of total RNA was mixed with formaldehyde, formamide, 10×MOPS buffer and DEPC-treated water. This mixture was heated to 65° C. for 10 min and gel loading buffer added (50% glycerol, 1 mM EDTA, 0.25% Bromophenol Blue, 0.25% Xylene Cyanol). Samples were then loaded onto a 1% agarose gel containing 5% formaldehyde and 1×MOPS buffer. RNA markers (0.28-6.58 Kb range) from Promega were used. The gel was run overnight at 30V with buffer recirculating. After electrophoresis, RNA markers were cut off, stained with ethidium bromide and photographed. The remaining gel was northern blotted as detailed in Sambrook et al. (supra). Membranes carrying RNA were prehybridised for an hour at 65°C. in hybridisation solution (6×SSC, 5×Denhardts, 0.5% SDS, 20 μg/ml salmon sperm DNA). One hundred and fifty ng of DNA (for probe) was labelled using the Amersham Multiprime kit and added to the membrane which was hybridised overnight at 65° C. The membrane was then washed three times at room temperature (2×SSC) for 10 min each Two further washes were done for 30 min in 0.1×SSC, 0.1% SDS. Membranes were then rinsed in 0.2×SSC, wrapped in Gladwrap and exposed to Fuji film for required time.

Expressed Sequence Tag Analysis

Individual, random, recombinant phage plaques were picked from the cDNA library, placed in 100 μl sterile water and boiled for 3 min before being put on ice. Five μl of this material was used as a template for a PCR reaction using primers FpB and RpB2 as described above. PCR products were then purified using the Qiaquick (Qiagen) PCR purification kit and cycle sequenced with the RpB2 primer and the aid of an ABI automated sequencer.

All DNA sequences were manually inspected and edited to remove vector sequences and sequences of poor quality normally close to the primer binding sites. The poly A tail, if present, was also removed and the N. caninum data set was then compiled using CreateDB into a local database (MyDB:N caninum) on the Australian Genome Information Service (ANGIS). BlastN was used to search MyDB:N caninum for sequences homologous to those under study. Matches were considered significant if scores were returned with a probability ≧10⁶.

Identification of Antigens

Immunoscreening of a tachyzoite cDNA expression library with serum from a naturally infected cow detected a wide range of positive plaques. The inventors have identified several new genes and gene fragments thereof including NcGRA2 (present Example), 24B (Example 2) and NcP20 (Example 3). Identification and characterisation for each of these new genes is described hereinbelow.

Isolation of GRA2-Like Sequences from % Enomic DNA of N. caninum

PCR was performed using total cellular DNA from NC-Liverpool and NC-SweB1 using primers 12F2 (5′CGAGCACCCACAAGTAA3′) (SEQ ID NO: 18) and 12R2 (5′GACCATAACGGATGCAAC3′) (SEQ ID NO: 19). PCR and DNA sequencing was also performed with primers P28F (5‘CAGCGGT’ TATTCCGGATA3) (SEQ ID NO: 20) and P28R (5′GCCTCAAGAATTTCCTCAGC3′) (SEQ ID NO: 21). PCR products were then purified using the Qiaquick (Qiagen) PCR purification kit and sequenced by cycle sequencing and the aid of an ABI automated sequencer.

GRA2 intron sequences were amplified by PCR using primers CRLF (5′GGTAGGTTACCACAACTTGC3′) (SEQ ID NO: 22) and CRIF (5′GCAATTGCATTGAGCATC3′) (SEQ ID NO: 23) that were designed from within the intron sequence of GRA2. The PCR cycling conditions used were; 95° C., 3 min, 1 cycle; 95° C., 45 sec; 50° C., 45 sec; 72° C., 1 min, 28 cycles; 72° C., 5 min, 1 cycle. Five μl of PCR product was run on a 1% agarose gel to check for amplification and size.

Expression of Gra2 in E. coli

The open reading frame (ORF) of GRA2 was PCR amplified from clone 12 with primers pTrcHisDA12F2 (5′ACGGATGGATCCGTTCACGGGGAAACGTTGG3′) (SEQ ID NO: 24) and pTrcHisIDA12R2 (5′ACGTCAGAATTCTAACGCCATACACACCGT3′) (SEQ ID NO: 25). These primers place unique BamH1 and EcoR1 restriction sites on the five and three prime sides of the GRA2 ORF respectively. The PCR product was checked on a 1% agarose gel for size and purified using a Qiaquick PCR purification kit. DNA from the purified PCR product and pTrcEisB vector (Invitrogen) were then digested with both BamHI and EcoRI restriction enzymes for three hours at 37° C. The digested DNA were purified using a Qiaquick column and checked on a 1% agarose gel. The ORF of GRA2 was then ligated into the pTrcHisB vector and transformed into E. coli DH5α. Individual recombinants were screened for inserts by PCR using primers pTrcHisFwd (5′GAGGTATATATTAATGTATCG3′) (SEQ ID INTO: 26) and pTrcHisIDA12R2. The sequence of the constructs made were confirmed by cycle sequencing. This strategy ensures the initiation codon of GRA2 is cloned in-frame into the pTrcEsB vector, which following transcription and translation should produce a polypeptide of 26 kDa. Subsequently, E. coli containing recombinant DNA were grown in LB medium containing ampicillin and at mid-log phase were induced with 1 mM IPTG. After several hours, the bacteria were collected by centrifugation and solubilised in guanidinium lysis buffer. His-tagged protein was purified using Ni-NTA (Qiagen) resin following the manufacturer's instructions for preparation of denatured E. coli cell lysate. Proteins were analysed on 14% SDS-PAGE gels by either staining with Coomassie blue or by western blotting after transfer to PVDF membrane (Atkinson er al. 1999). Antigen expression was detected using pooled, mouse sera from animals made resistant to a lethal challenge of NC-Liverpool. This serum was produced in female in-bred balb/C mice using two infections of NC-SweB1 tachyzoites as described by Atkinson et al (1999).

Secondary Structure Predictions for Gra2

Signal peptides were predicted using the SIGCLEAVE program of von Heijne (1986). The protein sequence of NcGra2 was also submitted to the PSA server and a secondary structure prediction made using a Type-1 analysis and the DSM model of Stultz et al. (1993) which presumes the protein is a monomeric, single-domain, globular, water-soluble protein. The following algorithms were subsequently used to investigate the location of potential helical structures in NcGra2: SSPRED (Mehta et al., 1995), nnSSP (Salamov and Solovyev, 1995), PHDsec (Rost and Sander, 1993; Rost, 1996), GOR 1 (Gamier et al. 1978), 2 (Gibrat et al. 1987) and 4 (Garnier et al. 1996), SIM:A96 (Levin, 1997), LEV (Levin et al. 1986), DPM (Deleage and Roux, 1987), predator (Frishman and Argos, 1996), SOPM (Geourjon and Deleage, 1994), and SOPMA (Geourjon and Deleage, 1995). Solvent accessibility was performed using PHDacc (Rost and Sander, 1994).

DNA Vaccination in Mice

Constructs were made using GRA2 cDNA and PCR in the following way. In order to clone GRA2 in the correct orientation (pGRA2), primers VR1012F (5′CGTACGTCTAGAGCCACCATGTTCACGGGGAAACGTTGG3′) (SEQ ID NO: 27) and VR1012R2 (5′ACGTCAGGATCCGCACGCACACAAAGCCCA3′) (SEQ ID NO: 28) were used to PCR amplify the open reading frame of GRA2. In this approach, an Xba1 site is placed upstream of a consensus Kozak sequence and the ATG start site At the 3′ end a BamH1 site is placed immediately downstream of the stop codon The resulting PCR product was purified using a Qiaquick (Qiagen) purification kit; cleaved with BamH1 and Xba1 (Promega) in multicore buffer for 3 hours. The restriction product was purified with the Qiaquick kit and ligated into BamH1/Xba1 doubly digested VR1012 (Vical). The ligation was transformed into E coli DH5α and kanamycin resistant colonies selected. Transformants were screened by PCR with primers VR1012Fwd (5′GCTGACAGACTAACAGACTG3′) (SEQ ID NO: 29) and VR1012Rev (5′AACTAGAAGGCACAGCAG3′) (SEQ ID NO-30) in order to identify colonies containing sequences of the correct size.

A similar procedure was used to construct a plasmid with GRA2 cloned in the reverse orientation (pRevGRA2). Primers Revp28F (5′CGTACGTCTAGAGCCACCATGGTCGGCGCCGCAGTCGTA3′) (SEQ ID NO: 31) and Revp28R (5′ACGTCAGGATCCTTCACGGGGAAACGTTGG3′) (SEQ ID NO: 32) were used to generate a PCR product that was then cleaved with BamH1 and Xba1. The product was cloned as above. The inserts of both pGRA2 and pRevGRA2 were sequenced to confirm the orientation of the inserts and the reading frame.

One hundred μg of VR1012 or recombinant VR1012 (in endotoxin-free TE, 10 mM Tris pH 8.0, 1 mM EDTA) carrying the N. caninum GRA2 gene in either forward (pGRA2) or reverse (pRevGRA42) orientations were injected, using a 30 gauge needle, into 6 week old, female in-bred Balb/C mice via either the pinna of the ear or intramuscularly into the footpad or leg (5 mice/group). All plasmids were maintained in E. coli DR5α and purified from 2.5 litre cultures (Luria-broth with kanamycin) using the EndoFree Plasmid Giga Kit (Qiagen). Changes in mean mouse body weight between days 14-27 post infection (dpi) with N. caninum tachyzoites were analysed by a one-factor-repeated measures analysis of variance, with treatment as the factor and time as the repeated measure. All the sampling times were included in the analysis, although mice which died or were euthanased before the fifth sampling time were excluded.

Infection of Pregnant Mice

Ovulation of 9 week old, female outbred Quackenbush (Qs) mice was synchronised using a single injection of folligon (Intervet) followed by a single injection of chorulon (Intervet) 48 hours later. Female mice were then mixed individually with a male stud for 24 hours and mating was detected by the presence of a vaginal mucoid plug. At day 8 of pregnancy, mice were injected subcutaneously with culture-derived tachyzoites of N. caninum. Pregnancies were allowed to proceed to day 21 when all mice were autopsied and serum taken.

The experimental groups were:

Cnp; a control group (10 mice) which were not pregnant;

1) non-pregnant mice (10) which were infected with 10⁶ tachyzoites of N. caninum (NC-Liverpool);

Cp, a control group (5) of un-infected pregnant mice;

2) pregnant mice (8) infected with 10⁷ tachyzoites of N. caninum (NC-Liverpool);

3) pregnant mice (8) infected with 10⁶ tachyzoites of N. caninum (NC-SweB1).

Enzyme-Linked Immunosorbant Assay (ELISA) Using NcGra2

Histidine-tagged, recombinant NcGra2 (purified on Ni-NTA resin as described previously) was coated onto a 96-well microtitre plate at a concentration of 1 μg/well diluted in ELISA buffer 1 (70 mM NaHCO₃, 29 mM Na₂CO₃, 3 mM NaN₃, pH 9.6). Following overnight incubation at 4° C., the plate was washed 3 times in wash buffer (0.15M NaCl, 0.3% Tween 20). Pooled, experimental serum samples from mice were diluted 1:100 using phosphate buffered saline (PBS) and 100 μl of each sample was added to the plate in duplicate. The plate was incubated for 2 hours at 37° C. and then washed as before. One hundred μl of biotinylated antibody to mouse IgG1 or IgG2a (The Binding Site, UK) was added to each well at a dilution of 1:6000 in ELISA buffer 2 (0.5 g bovine haemoglobin, 0.3% Tween 20, 3.1 mM NaN₃, pH 7.2 in PBS). Following a 2 hour incubation at 37° C., the plate was washed and each well coated with 100 μl of Extravidin alkaline phosphatase (Sigma, USA) at a dilution of 1:5000 in ELISA buffer 2. After incubation for 1 hour at 37° C. the plate was again washed and 100 μl of Alkaline Phosphatase Substrate 104 (Sigma, USA) was added at a concentration of 1 mg/ml in ELISA buffer 3 (58 mM NaHCO₃, 42 mM Na₂CO₃, 2 mM MgCl_{2.6}H₂O, pH 9.8). The plate was incubated at 37° C. for 30 min, allowing sufficient colour development. The absorbance reading of each well at 405 nm was determined using an electronic plate reader (Biorad).

RESULTS

Isolation of NCGRA2

Twenty-five independent bacteriophage clones were isolated that expressed antigen which is recognised by antibody from a cow that was chronically infected with Neospora. All were sequenced using ABI sequencing technology. Several of these were found to bear DNA sequence homology to the Nc4.1 (eight) and Nc2.1 (two) recombinant clones described by Lally et al. (1996) and were not studied further. The sequence of another recombinant (clone 12) was found to predict significant protein sequence homology of the gene product to the amino acid sequence of the 28 kDa antigen (Gra2) of T. gondii (Prince er al. 1989; hereafter called IgGra2) and so was studied further. The sequence of clone 12 (hereafter called NCGRA2) clustered using the Tblast X algorithm, in the ToxoDB database with the cluster Ctoxqual2_{—}1721 and Ctoxqual2_{—}289 which contains sequences coding for Gra2. Thus the present inventors concluded that this clone represented a N. caninum gene which has not been described previously.

Expression and Gene Organisation of GRA2 in Tachyzoites

RNA was extracted from tachyzoites of NC-Liverpool and subjected to northern blotting using clone 12 as probe. A single transcript of approximately 1300 bp was detected. DNA sequence from 522 ESTs was generated and 12 of the data set were homologous to NCGRA2. This represented the most abundant transcript detected in the data set and corresponds to a level of expression of approximately 2.3%. The EST sequences and the sequence of clone 12 were compiled to yield a consensus sequence for the mRNA of NCGRA2.

PCR amplification of total cellular DNA using primers 12F2 and 12R2 from both NC-Liverpool and NC-SweB1 yielded two PCR products (approximately 800 and 1200 bps). These were both sequenced and subsequent BlastN searches revealed the 1200 bp fragment contained the desired GRA2-like sequences The 800 bp fragment was found to be homologous to cytochrome B of T. gondii (GenBank accession number AF023246). The 1200 bp product from NC-SweB1 and NC-Liverpool was almost identical in sequence (98%).

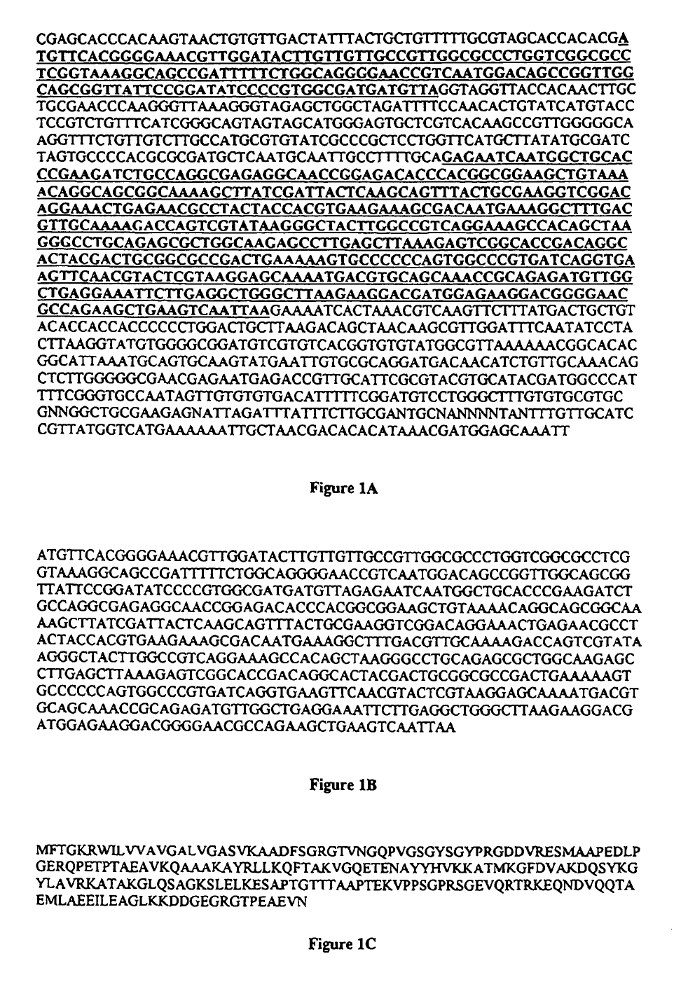

Genomic and cDNA sequences for NCGRA2 were compared. The gene structure possessed 2 exons separated by an intron of 241 bp (FIG. 1A). The intron showed no sequence similarity to any sequence in GenBank including the intron of the T. gondii gene. In order to confirm this observation, PCR was performed with primers CRIF and CRIR using both N. caninum and T. gondii genomic DNA as template. A PCR product of 228 bp was produced only from N. caninum DNA (NC-SweB1; NC-Liverpool and NC1 strains) but not from DNA of Vero or T. gondii (RH or Beverley strains). DNA sequencing confirmed the PCR product was derived from the N. caninum intron

A comparison of the N. caninum and T. gondii (N993921) coding sequences revealed (excluding the three prime end) a 56% sequence similarity between them. The nucleotide differences between the two sequences were manifest as a range of indels and nucleotide substitutions. In addition, the three prime end of NCGRA2 encoded 19 additional amino acids not present in IgGra.

Expression of NCGRA2 in E. coli

Western blotting detected a 45 kDa antigen in both soluble and insoluble denatured, reduced extracts of E. coli. Consequently, bacteria expressing NCGRA2 were collected by centrifugation and solubilised in guanidinium lysis buffer and His-tagged protein purified using Ni-NTA resin following the manufacturer's instructions. After purification, Coomassie blue staining of an SDS-PAGE revealed the presence of a 35 kDa protein that was also detected specifically by mouse anti-N caninum antisera Injection of this protein into mice, resulted in the production of ISG antibodies to an N. caninum tachyzoite antigen of 29 kDa in NC-SweB1 and NC-Liverpool.

Secondary Structure Predictions

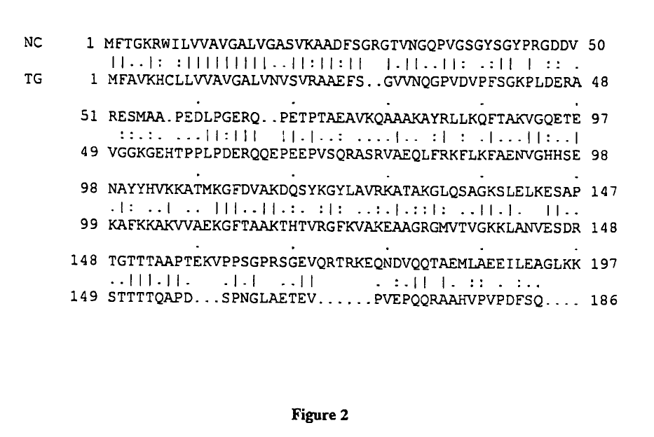

Analysis of the predicted protein sequence encoded by NCGRA2 revealed the amino acid sequence was 52% similar to IgGra2 (FIG. 2). The amino terminus was particularly conserved. The SIGCLEAVE program predicted a signal peptide (WILVVAVGALVGA) (SEQ ID NO: 33) in this region that was almost identical to that present in T gondii. Mercier et al. (1993) predicted that the secondary structure of Gra2 in T. gondii was a globular protein with two amphipathic helices separated by a 10 amino acid linker. Consequently, the protein sequence of NcGra2 was submitted to the PSA server and a secondary structure prediction made using a Type-1 analysis and the DSM model of Stultz et al. (1993). The analyses showed that NcGra2 most probably belongs to the protein superclass which predominantly contain alpha helices (probability 0.95574). The algorithm also predicted the presence of two major and one minor helical regions in NcGra2 and that the most plausible explanation for the structure of the remaining residues of NcGra2 was in the form of loops or turns. In NcGra2 the helical regions spanned residues 70-110) 110-150 and 170-190. This secondary structure prediction was investigated further using 14 additional algorithms. A consensus derived from this alignment provides considerable support for four, and not three, helices (H1-4) spanning residues T70-V92, E95-D116, K120-G132 and N177-G194. The amphipathic nature of H1, H2 and H4 is clearly evident by the distribution of hydrophobic and hydrophilic residues on alternative sides of the helical wheel.

Since the predictions for the location of H1 and H2 were similar to but not identical to that for IgGra, the predictions of Mercier et al. (1993) were re-evaluated in light of knowledge gained from NcGra2. H1 in IgGra2 was assigned to residues P70-V94, H2 to S97-K115 plus an additional helix (H3) was predicted at R1121-G33. Thus these results differ from those of Mercier et al. (1993) by the location of the start of H1 at P plus the existence of H3 not previously identified nor suggested. Proline has long been known to be a “helix breakers” (Chou and Fasman, 1973) and so is generally restricted to the first turn of the N-terminal helix. Its location as the first residue in an amphipathic helix, or in a turn leading into the helix, is suggested by the fact that proline has no free NH group and therefore cannot form the conventional intra-helical NH . . . O═C hydrogen bond (Chakrabarti and Chakrabarti, 1998). The subtle differences suggested here in the helical structures from those reported by Mercier et al. (1993) results from the use of a more extensive number of refined algorithms which exist and are in use today.

DNA Vaccination

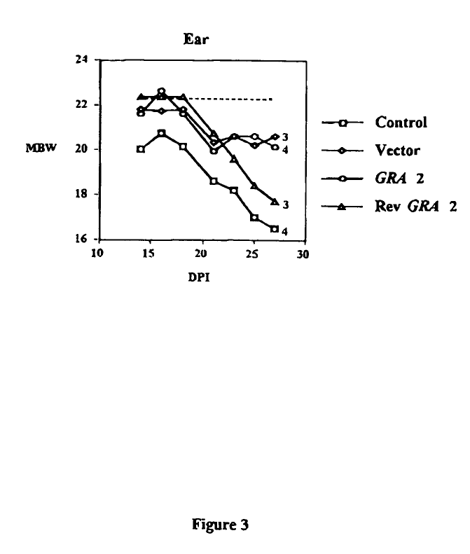

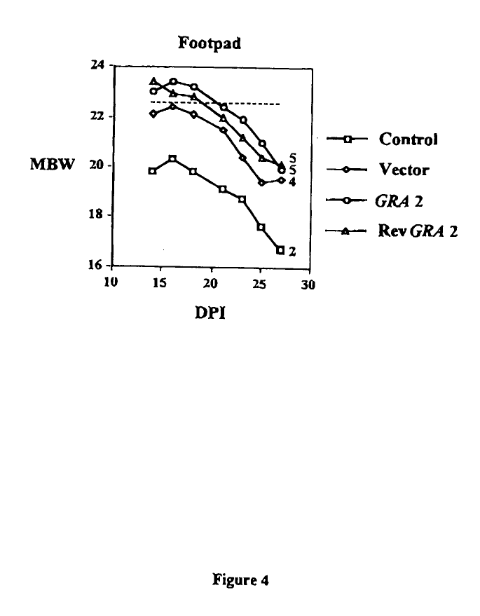

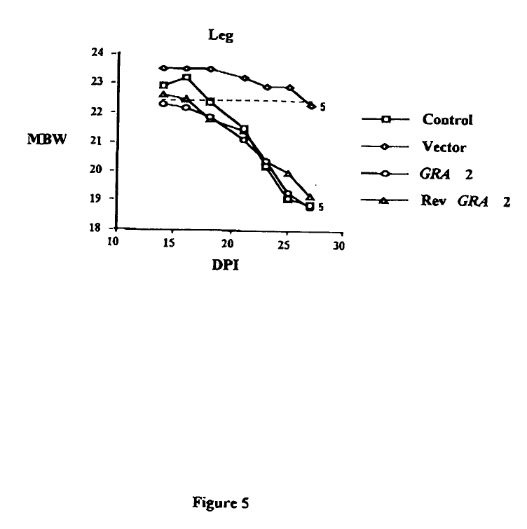

Since the route of administration of DNA vaccines may effect the outcome of vaccination groups of mice were given pGRA2 via either the pinna of the ear, or intramuscularly via the footpad or leg and subsequently challenged with N. caninum tachyzoites. The results are shown in FIGS. 3 to 5. Mice which were not immunised nor challenged with N. caninum showed little change in body weight between 14-27 dpi ((+)0.1 g). In addition, control mice which were sham treated, along with mice which were not immunised but were challenged with N. caninum showed a very large drop in mean body weight (−3.4 to −4.6 g) along with clinical signs of neosporosis (predominantly a ruffled appearance with a limited level of limb paralysis).

In the group immunised via the pinna, mice receiving pRevGRA2 rapidly lost weight between 14-27 dpi. Three mice were euthanased because of the advanced signs of neosporosis. In contrast, the groups receiving either VR1012 or pGRA2, all mice lost weight between 14-20 dpi when 3 of the mice became unwell and were euthanased. However, the other 7 mice remained well, although ruffled, and maintained their body weight in both groups. Analyses of variance (Table 2) confirmed these treatments were significantly different from the other two groups.

TABLE 2
Analyses of variance for mouse weights

| | Probabilities for Treatment Groups | | |
|---|---|---|---|
| Source | Ear | Footpad | Leg |
| Treatment | 0.046 | 0.217 | 0.222 |
| Time | <0.001 | <0.001 | <0.001 |
| Treatment*Time | <0.001 | <0.001 | <0.001 |

In the experiment involving footpad immunisation, all three groups of mice receiving plasmid DNA (VR1012, pGRA2 and pRevGRA2) all showed some weight loss between 14-27 dpi but this was significantly less than the control group. Although all mice became ruffled, mortality was limited to 1 animal in the DNA vaccinated groups compared to ⅗ in the control group. Statistical analysis of the changes in mean body weight described cons that mice immunised via the footpad with plasmid or recombinant DNA were significantly different from the control group, although the mean weight loss in all groups at the end of the experiment was significantly less than mice which were not immunised nor challenged.

An experiment was also performed where the plasmid DNAs were delivered intramuscularly via the hindleg. Only the group receiving VR1012 retained their body weight over the course of the experiment and this was statistically significant. Although there were no mortalities in this experiment, all mice in the remaining three groups were ruffled and showed rapid weight loss over the course of the experiment. No doubt if this experiment bad been allowed to continue these mice would have died.

In summary, immunisation of mice via the footpad with VR1012 or pGRA2 demonstrated evidence for partial protection against mortality due to neosporosis in this model. Protection against weight loss due to N. caninum infection was also demonstrated in both pinna and footpad delivery experiments, although it was more pronounced when the plasmid DNA was delivered via the footpad.

Use of NcGra2, Expressed and Purified from E. coli, to Detect Antibodies Against N. caninum in an ELISA Assay

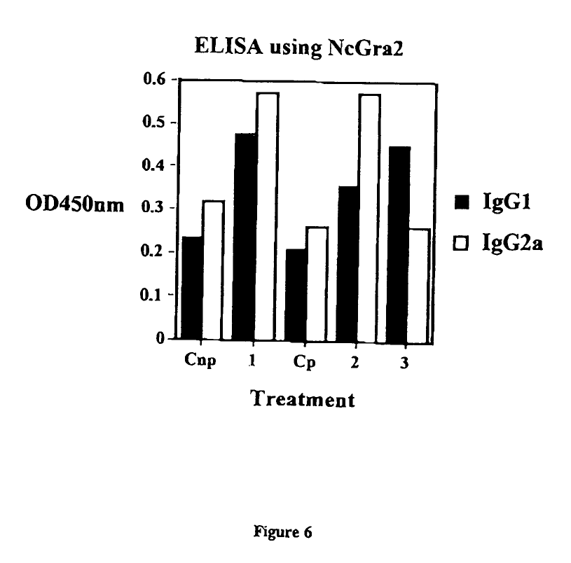

Injection of 10⁶ tachyzoites of N. caninum (NC-Liverpool) into the non-pregnant Qs mouse induced no weight loss and no signs of clinical symptoms of neosporosis. An ELISA performed using NcGra2 purified from E. coli demonstrated that infection induced a strong IgG1 and IgG2a antibody response to this protein in these animals (compare the results of groups Cnp and 1 in FIG. 6). Experiments with pregnant Qs mice showed that an antibody response of similar magnitude was also induced by 10⁷ (group 2) tachyzoites of N. caninum (NC-Liverpool). Animals in this group infected with NC-Liverpool delivered live pups (total live pups born=58; mean litter size=8) with only 5 stillborn. In contrast, infection of mice with NC-SweB1 produced only 18 viable pups. Histopathology demonstrated extensive foetal resorption in this group. ELISA with NcGra2 demonstrated mice infected with NC-SweB1 possessed a larger IgG1/IgG2a antibody ratio to this protein than those mice infected with NC-Liverpool (group 3)

In summary, this study has shown that an ELISA assay, using NcGra2, expressed and purified from E. coli, can be used to detect antibodies produced during infection of an animal by N. caninum. Furthermore, detection of specific antibody sub-types induced during pregnancy against NcGra2 (in this example, IgG1 or IgG2a, or more specifically the ratio of IgG1/IgG2a) may provide a method of predicting the outcome of infection during pregnancy (e.g. whether foetal resorption has/will occur and whether young may be born live).

DISCUSSION

A gene from N. caninum, homologous to the GRA2 gene of T. gondii, has been cloned and sequenced. Both N. caninum and T. gondii genes are composed of two exons and a single intron and are highly expressed in tachzyoites (as detected by northern blotting and EST sequencing) as a very abundant messenger RNA.

Early research on the T. gondii antigen showed it to have a submembraneous location in the tachyzoite although more recent work has now demonstrated that the TgGra2 protein is located in the dense granules of the tachyzoite. Upon infection TgGra2 is secreted into the parasite-containing vacuole where it is rapidly and specifically targeted to a network of membranous tubules which connect with the vacuolar membrane. The subcellular location and function of NcGra2 is currently not known, however, it is likely to fulfil a similar function to TgGra2. Although the protein sequence of NcGra2 is only 52% similar to TgGra2, the secondary structure predictions made, using a wide variety of algorithms, indicate a high degree of support for both proteins containing several amphipathic helices separated by loops and turns. Thus although the present results show that the protein sequence of Gra2 is not highly conserved, it would appear maintenance of secondary structure has occurred during the evolution of these molecules. Sufficient dissimilarity exists, however, between the T. gondii and N. caninum proteins for us to hypothesise that they are antigenically distinct. For example, the carboxy termini differs between NcGra2 and TgGra2. This region contains, in T. gondii, an epitope recognised by antibodies from naturally infected humans

Expression of the entire GRA2 ORF in a prokaryotic expression vector (the plasmid pTrcHisB) was achieved. A purification procedure was used to isolate the recombinant protein, of apparent molecular weight 35 kDa, from E. coli. The molecular weights reported here are somewhat anomalous, because predictions of the protein size from the open reading frame for the bacterially expressed protein (including vector encoded amino acids) suggest a size of 26, and not 35 kDa The anomalous mobility may simply be the influence of the proteins shape because two or three helices were predicted, by many different types of computer algorithms, to exist in the secondary structure of this protein. Although post-translational modifications, such as glycosylation, have been shown to occur in T. gondii such modifications were discounted because they do not occur in E coli. Despite the anomalous mobility, the purified recombinant protein maintained its antigenicity as determined by western blotting with sera from mice immune to neosporosis.

Extensive evidence now indicates that the route of delivery of a DNA vaccine may effect the outcome of the immunisation process (reviewed by Cohen er al. 1998). Injection of DNA into the pinna or intramuscularly via the footpad or leg was investigated because of the requirement to induce a Th1 response which is probably the basis of protective immunity against N. caninum infections. DNA vaccination into the pinna, footpad and leg have all been shown to be an effective way of inducing such a response in other situations. Surprisingly, it was shown that mice immunised with DNA into either of these three different sites gave a different outcome when challenged with N. caninum. Injection of VR1012 or pGRA2 into the pinna or footpad induced a significant level of partial protection against weight loss in the CNS model used. That the plasmid VR1012 induced partial protection on its own, irrespective of injection location, suggests that adjuvant activity supplied by the vector alone is an important constituent of the immunity demonstrated here. The nature of this activity is thought to result from the presence of immunostimulatory sequences, such as CpG motifs, in the vector leading to a preferential induction of a Th1 response.

Systems for the stable introduction of recombinant DNA (transformation) into parasites such as T. gondii and N. caninum have been developed. Several strategies such as chloramphenicol selection, complementation of tryptophan auxotrophy, pyrimethamine resistance and bleomycin resistance have been used to achieve stable transformation.

These systems can now be exploited for the homologous and heterologous expression of genes (Howe et al. 1997). In addition, the creation of “knock-out” mutants is considered the state of the art at this current time and provides a method for attenuating wild-type organisms in order to create novel live vaccines (Soldati et al. 1995). Knock-out mutants may be created by placing NCGRA2 sequences onto either side of a selectable marker, that upon transformation into N. caninum tachyzoites, will integrate into genomic NCGRA2 and “knock-out” endogenous expression. Changes in gene expression such as this ultimately may lead to the creation of novel lines of N. caninum that are attenuated in their ability to cause disease. Such mutant lines therefore have the ability to act as both live and killed vaccines against neosporosis. It will be appreciated that the nucleic acid molecules according to the present invention would be suitable candidates for the development of knock out mutants of N. caninum.

### EXAMPLE 2-24B

Materials and Methods

Identification of Clone 24B

Clone 24B was isolated from a tachyzoite cDNA library by immunoscreening with serum from cow X naturally infected with Neopora as described above.

The cDNA sequence of 24B was compiled from 2 sources. In the first instance PCR primers FpB/RpB2; 24BconF (5′ACCGTGGCAGTCCGCTGT3′) (SEQ ID NO: 34)/24BconR (5′TGGGCTGATGACCCCGTC3′) (SEQ D NO; 35); 24BconF/24BconR2 (5′CCAAGGCAGGAGAGGCAC3′) (SEQ ID NO: 36); 24BconF2 (5′ACCACTGCTCAACTAC3′) (SEQ ID NO: 37)/24BconR; and 24BconF3 (5′GCGCGTCTAGATAGCA3′) (SEQ D NO: 38)/24BconR were used to PCR template derived from plaque 24B directly or a PCR product derived from plaque 24B using primers FpB/RpB2.

Sequencing of 24B Genomic DNA

Genomic DNA was prepared by standard procedures involving lysis of tachyzoites in buffer containing 1% SDS, 10 mM Tris pH 9.0, 100 mM EDTA and proteinase K at 55° C. for two hours, followed by phenol chloroform extraction. The aqueous phase containing DNA was dialysed overnight at 4° C. in 10 mM Tris pH 8.0, 100 mMEDTA. After further phenol chloroform extractions the DNA was dialysed against 10 mM Tris pH 8.0, 1 mM EDTA at 4° C. for several hours.

Using the cDNA sequence derived for 24B, a variety of primers were designed (Table 3), which in various combinations were used to PCR and sequence the genomic DNA encoding the 24B mRNA. Each PCR product was sequenced several times in each direction to give a consensus sequence for the 24B genomic DNA.

The primers 24BconF/24BconR gave multiple bands when used to PCR genomic DNA and so were not studied further.

TABLE 3
Primers used to PCR amplify and
sequence the 24B gene from N. caninum

| | | | SEQ |
|---|---|---|---|
| Primer Name | Direction* | Sequence (5′ to 3′) | ID NO: |
| 24BconF | F | ACCGTGGCAGTCCGCTGT | 34 |
| 24BconF2 | F | ACCACTGCTCAACTAC | 37 |
| 24BconF3 | F | GCGCGTCTAGATAGCA | 39 |
| 24BconF4 | F | AGCCTATCTCTGCGTA | 40 |
| 24BconF5 | F | AGCTGACCACCTCACCGAT | 41 |
| 24BconF6 | F | TGAAGTCCCAAGCGTCCTC | 42 |
| 24BconF7 | F | ACTCTCCGTCTCTCTCTGC | 43 |
| 24BconR | R | TGGGCTGATGAACCCGTC | 35 |
| 24BconR2 | R | CCAAGGCAGGAGAGGCAC | 36 |
| 24BconR3 | R | CCACGCCCTGAACTGACT | 44 |
| 24BconR4 | R | GCCTTGTTGAGGATGGA | 45 |
| 24BconR5 | R | TGCTGGATCGAAGAC | 46 |
| 24BconR6 | R | AGGCGGGTAAATGGTAA | 47 |

*F, forward; R, reverse

Cloning and Expression of 24B into pTrcHisB

The open reading frame (ORF) of 24B was PCR amplified from cDNA clone 24B with primers 24BORF2-pTrcF (5′ACGCATGGATCCGGATCCTAAAGTGGAGAGT3′) (SEQ ID NO: 48) and 24BORF2-pTrcR (5′ACGTATGAATTCCCAAGAGGAAAACAATGT3′) (SEQ ID NO: 49). These primers place BamH1 and EcoR1 restriction sites on the five and three prime sides of the 24B ORF respectively. The PCR product was checked on a 1% agarose gel for size and purified using a Qiaquick PCR purification kit. DNA from the purified PCR product and pTrcHisB vector (Invitrogen) were then digested with both BamHI and EcoRI restriction enzymes for three hours at 37° C. The digested DNA were purified using a Qiaquick column and checked on a 1% agarose gel. The ORF of 24B was then ligated into the pTrcHisB vector and transformed into E. coli DH5α. Individual recombinants were screened for inserts by PCR using primers pTrcHisFwd (5′GAGGTATATATTAATGTATCG3′) (SEQ ID NO 50) and 24BORF2pTrcR. The sequence of the constructs made were coined by cycle sequencing. This strategy results in the cloning of the 24B ORF (minus the initiation codon) in-frame into the pTrcisB vector, which following transcription and translation should produce a polypeptide of 27 kDa. His-tagged 24B was purified from E. coli as follows. E. coli containing recombinant DNA were grown in LB medium containing ampicillin and at mid-log phase were induced with 1 mM IPTG. After 3 hours, the bacteria were collected by centrifugation and solubilised in lysis buffer containing lyzoyme. Cells were then sonicated until disrupted, passed through a 19.5 gauge needle, and then cleared by centrifugation for 15 min at 10,000 g. 24B was then purified from the remaining soluble fraction by Ni-NTA chromatography following the guidelines provided by the manufacturer (Qiagen). Briefly, lysate/resin mixes were allowed to mix for 1 hour on a rotary wheel, after which they were combined into a column The column was washed extensively in wash containing 20 and then 50 mM imidazole. Protein was eluted from the column in 70 and 100 mM imidazole solution and dialysed extensively against 0.9/o saline.

Cloning of 24B into pET25b

Using 24B cDNA as template, PCR was carried out with primers pET25-24BORF2F (5′-ACGCATGAATTCTATGGATCCTAAAGTGGAGAGT-3′) (SEQ ID NO: 51) and pET25-24BORF2R2 (5′-CATGACCTCGAGGACGCGCGGAACACCGTA-3′) (SEQ ID NO: 52) using PCR cycling conditions as follows 94° C.×2 minis 1 Cycle; 94° C.×45 sec, 50° C.×45 sec, 72° C.×1.5 mins 28 cycles 72° C.×5 mins 1 cycle. The PCR product was then purified using Qiagen PCR purification kit and 1 μl run on a gel to check concentration. PCR product was then digested at 37° C. for 3 hours with EcoRI and XhoI. The restriction digest was then purified with the Qiagen kit before ligation. Ligation was performed overnight at 4° C. with EcoRI/XhoI cut pET25b vector. Ligation was transformed into Top10 competent cells and transformations plated overnight. Recombinant colonies were streaked out and screened using vector based primers and PCR (T7 (T7P) promoter 5′TTAATACGACTCACTATAGGG3′ (SEQ ID NO: 53) and T7 (T7T) terminator primers 5′GCTAGTTATTGCTCAGCG3′) (SEQ ID NO: 54). These were used to assess the size of the insert.

A selection of colonies that appeared to have correct insert size were then sequenced to check that the insert had ligated in correctly.

E. coli (containing recombinant pET25b) from 50 ml L-broth cultures were pelleted and resuspended in 4 ml of lysis buffer (50 mM NaH₂PO₄, pH 8.0, 300 mm NaCl, 10 mm imidazole). Cells were lysed with lysozyme and sonication and cell debris removed by centrifugation at 3500 rpm, 4° C. for 10 min. Recombinant protein was bound by mixing with 5 ml of Ni-NTA resin for 1 hr at 4° C. which was then transferred to a 5 ml disposable column (QIAGEN) and the resin allowed to settle before draining Contaminating proteins were removed by washing the column with 8×5 ml washes with 50 mM NaH₂PO₄, pH 8.0, 300 mm NaCl, 20 mM imidazole, 2×5 ml washes with 50 mM NaH₂PO₄, pH 8.0, 300 mm NaCl, 50 mM imidazole and 2×2.5 ml washes of 50 mM NaH₂PO₄, pH 8.0, 300 mm NaCl, 70 mM imidazole. Protein was eluted off in 2×2.5 ml elutions of 50 mM NaH₂PO₄, pH 8.0, 300 mm NaCl, 100 mM imidazole and 1×2.5 ml elutions of 50 mM NaH₂PO₄, pH 8.0, 300 mm NaCl, 250 mm imidazole. Elutions were checked by SDS-PAGE and dialysed against 0.9% saline. The amount of protein recovered was estimated by assaying with Bradford reagent (Biorad) and then the protein was lyophilised and stored at −20° C.

Generation of Antiserum

Mouse antibodies were prepared against recombinant p24B. 1 μg of protein was mixed with VSA-3 adjuvant (Novartis) and injected subcutaneously into five female Qs mice (6 weeks of age). 14 days later a small amount of blood was removed from the tail vein of each mouse and used in western blotting.

Western Blotting

N. caninum tachyzoites were recovered from in vitro culture and reduced to protein extracts by resuspension in lysis buffer and disruption by Sonication at 50W/20 KHz for 10-20 secs. The equivalent of 10⁷ tachyzoites were diluted 1:1 in loading buffer with (reduced) or without β-mercaptoethanol (non-reduced), boiled for 3 min and then loaded onto a 15% Tris glycine acrylamide get and electrophoresed at 100 v. After electrophoresis, proteins were transferred to PVDF membrane in a Tris/glycine/methanol buffer at 300 mA for 2 hrs. Following transfer, proteins were visualised with Ponceau S stain and the position of the Biorad low molecular weight range markers identified. The membrane was allowed to dry before being cut into 0.5 cm strips for immunoblotting. Membrane was then re-wet and blocked in 5% skim milk in PBS/0.03% Tween for 30 min at room temperature. Strips were washed for 3×10 min in PBS/Tween and then incubated in the first antibody diluted to 1:100 in 5% skim milk in PBS/Tween at room temperature for 1 hr. The strips were washed for 3×10 min in PBS/Tween then incubated in anti-mouse IgG-alkaline phosphatase conjugate diluted 1:1000 in 5% skim milk in PBS/Tween at room temperature for 1 hr. Strips were again washed for 3×10 min in PBS/Tween then antigen-antibody complexes were detected by incubation of the strips in NBT/BCIP substrate. Molecular weights of visualised bands were estimated using the molecular weight markers.

Results and Discussion

Characterisation of 24B

Immunoscreening of a tachyzoite cDNA expression library with serum from a naturally infected cow detected a wide range of positive plaques. One of these plaques was subsequently called 24B.

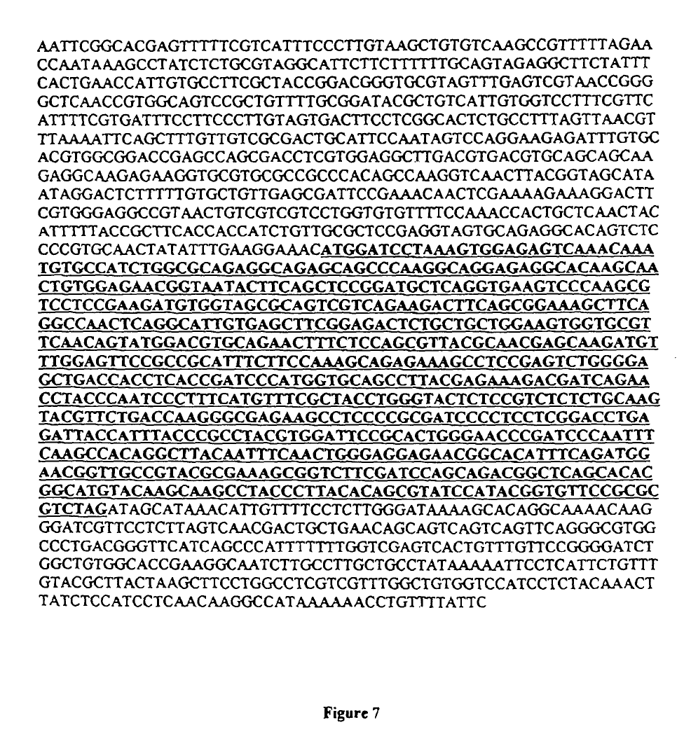

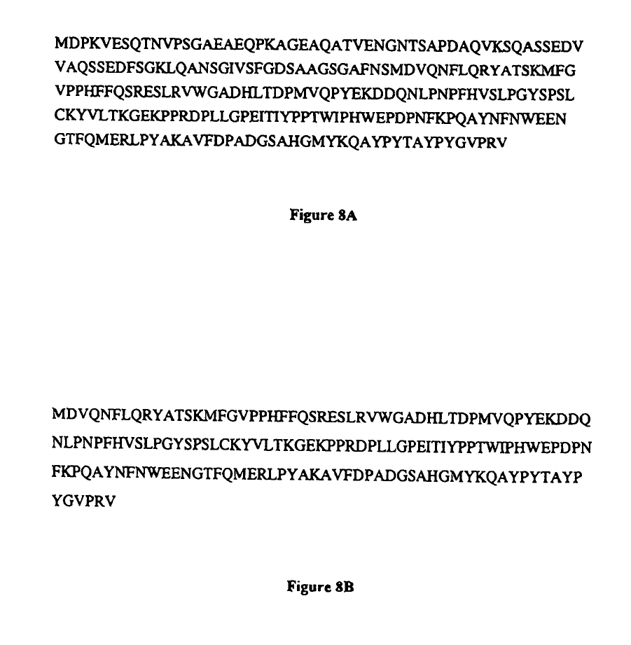

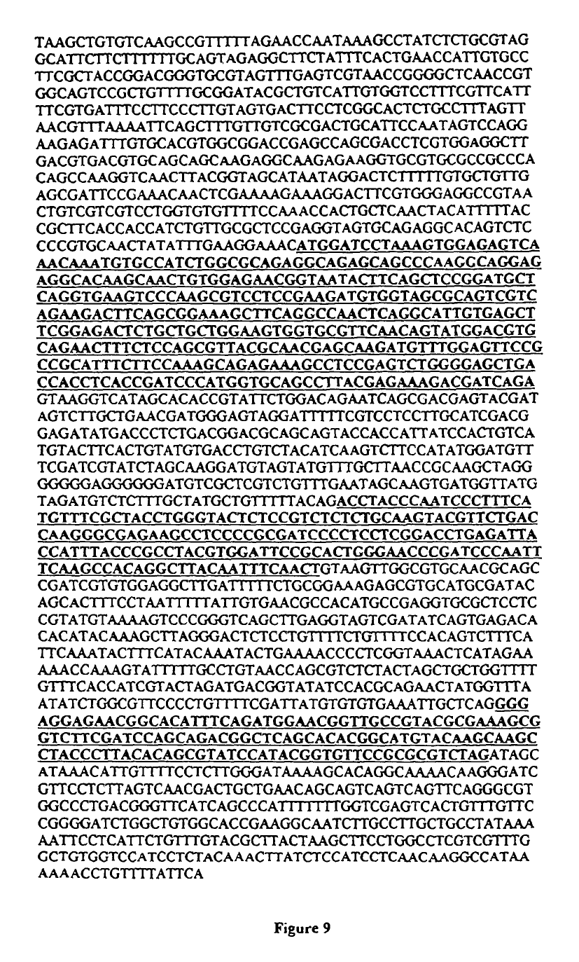

The sequence compiled for the 24B cDNA was 1744 bp long (plus a poly A tail of 47 residues) (FIG. 7) and contained a number of potential open reading frames (ORFs). However homologies detected to T. gondii and E. tenella sequences by database searching pointed to the ORF being located between positions 628 and 1420. This ORF was 729 bp long encoding a 27 kDa polypeptide containing 243 amino acids (FIG. 8A). The first ATG codon in the 24B gene is located at 628 bp and is believed to be the start codon of this gene as it is in a favourable context for initiating translation and there are no other ATG codons upstream. The sequence of the 24B gene is provided in FIG. 9.

A BlastP search of the NR Nucleic Acid database with the polypeptide encoded by the 24B cDNA revealed matches to RNA polymerases from a wide variety of taxa. Further inspection revealed these matches were to proline rich regions only, and the matches were otherwise not meaningful. Analysis of the predicted 24B polypeptide showed proline (11.2%), alanine (9%) and serine (8.7%) were the most abundant amino acids present. Further, analysis of the 24B amino acid sequence revealed a high number of short repetitive sequences. Of note are the AYPY repeats near the carboxy terminus, and the serine/glutamine associated, imperfect repeats in the first half of the polypeptide. The ORE encoding the polypeptide contained just three repeats greater than 7 bases long, and these were AGAGGCA, ACTGGGA and CAATTTCAA.

The 24B polypeptide sequence was also scanned against the Prosite database in order to identify protein motifs. Two ASN glycosylation sites and two N-myristilation sites were located, however a larger number of protein kinase C and casein kinase II phosphorylation sites were detected. No significant signal sequences was detected using the SIGCLEAVE program.

Secondary structure predictions for the 24B polypeptide demonstrated most of the residues were likely to be present in a random coil, although several helical structures were predicted.

The 5′ untranslated region was 627 bp long, while the 3′ untranslated region was 324 bp long. Comparison of cDNA (56% GC) and genomic sequences revealed the presence of two introns (332 and 422 bp; 47 and 44% GC respectively) and three exons in the gene sequence. Both introns obey the GT-AG rule in that they both start with the GT dinucleotide, and finish with the AG dinucleotide.

A BlastN search of the NR Nucleic Acid database with the 5′ untranslated region from the 24B cDNA sequence revealed sequence similarities (67% over 50 bases) to those found in the 3′ untranslated regions of cytokine-like genes of mammals such as MIP-1α (HSMIP1A) and LD78 (HSLD78A). No other similarities were found m this database at this time. For example, a BlastN search of the NR Nucleic Acid database with either of the intron sequences nor the 3′ untranslated region found no matches to sequences in the database.

A TBlastX search of Apicomplexan databases with the entire 24B cDNA sequence revealed matches to several ESTs from T. gondii. Specifically, four ESTS were found by Blast searching the ToxoDB which possessed sequence similarity to 24B from N. caninum. Two of these (W63156 and AW702928) aligned with the 5′ end of the N. caninum 24B sequence, whereas another two (N82407 and W63085) aligned at the 3′ end. Hence, it was concluded that a homologue of 24B clearly exists in T gondii.

Expression of 2413 in E. coli

Expression of the N. caninum 24B ORP in the plasmid vector pTrcHisB was achieved using the strategy described above, however the yield of protein obtained was poor from standard broth cultures. Recloning of the sequence into pE125b resulted in much higher levels of 24B protein expression, which could be purified by Ni-NTA chromatography. Antisera raised in mice to this protein, detected weakly a similar sized native antigen in N. caninum tachyzoites.

### EXAMPLE 3

### NcP20

Materials and Methods

Parasite Culture

N. caninum isolates NC-Liverpool (Barber et al. 1995) and NC-SweB1 (Stenlund et al. 1997) were propagated in vitro in Vero host cells according to established procedures (Barber et al. 1995).

Immunoblotting

Female Balb/C mice were made resistant to an acute, lethal infection of NC-Liverpool by 2 infections of NC-SweB1 as described in (Atkinson et al. 1999). Immunoblotting was used to compare antibody responses of these resistant mice and a separate group of acutely infected, naive mice (Atkinson et al. 1999).

Affinity purified antibodies (APAbs) were prepared (Hemphill et al. 1997b) by immunoblotting 100 μg of NC-Liverpool antigen separated by SDS-PAGE onto PDVF. A portion of the PDVF was cut out from either side of the PVDF membrane and a section covering the 15-30 kDa size range were visualised by immunoscreening. The portions were then realigned with the main strip of PVDF and the region spanning antigens 15-30 kDa excised. Polyclonal antibody from the resistant mice (n=5, pooled) was then bound to the excised PVDF for 6 hours at RT at a dilution of 1/10. The polyclonal antibody was removed and the antigen strip was washed thoroughly in Tris-buffered saline/Tween (IBS-Tween) 3 times for 20 mins. Bound antibody was eluted in a low pH buffer (50 mM Trio 50 mM Glycine; pH 2.8) for 5 mins at RT. After neutralisation with 1/10 volumes of 1 M Tris, the eluate was diluted with TBS and 5% skim milk powder to a final volume of approximately 10 ml for use in immunoscreening a cDNA library.

cDNA Library Construction

The cDNA expression library described in Example 1 was used in this study.

Immunoscreening cDNA Library

The APAb was used to screen 10,000 clones of the cDNA expression library described in Example 1 following standard immunoscreening procedures (Sambrook et al. supra). The clones were adsorbed to IPTG impregnated PVDF membranes (2 h, RT). Membranes were then probed with the APAb (45 min. RT). After washing (3×10 min) in TBS-Tween the membranes were placed in anti-mouse IgG (Sigma) diluted in 1/1000 in TBS and 5% skim milk powder (45 min, RT). Washing was repeated and development took place in alkaline phosphatase buffer containing nitroblue tetrazoleum and 5-bromo-4-chloro-3-indolyl-phosphate (Sambrook et al., supra), Positive clones were rescreened until plaque pure.

Characterisation of cDNA Clones

Plaque pure positive clones were picked, placed in 100 μl sterile water, boiled and subjected to PCR and sequencing (Ellis et al. 2000). Sequences were blasted (BlastN and TblastX) using the Australian Genome Information Service (ANGIS) against the GenBank or NR Nucleic Acid database. This latter database is compiled by ANGIS and contains non-redundant data from GenBank, EMBL and PDB Matches were considered significant if scores were returned with a probability greater than 10⁶. DNA sequences were also blasted against the Toxoplasma Database of Clustered ESTS (ToxoDB; http://www.cibil.upenn.edu/agi-bin/ParaDBs/Toxoplasma/index.html) (Kissinger et al., 2003).

Protein Structure Predictions

The protein sequence of NcP20 was submitted to the PSA server (http://bmerc-www.bu.edu/psa/) and a secondary structure prediction made using a Type-1 analysis and the DSM model of Stultz et al. 1993) which presumes the protein is a monomeric, single-domain, globular, water-soluble protein.

Production and Immunisation of a Fragment NcP20 in E. coli

Expression of a Fragment of NcP20 in E. coli

Since there was three potential start codons in the mRNA encoding NcP20, the open reading frame (ORF) of NcP20 was PCR amplified from EST clone P06 with either

| | |
|---|---|
| P20-ATG1F (5′ACGTATGGATCCGTTTTGTCAGGTGTTCTTG3′); | (SEQ ID NO: 55) |
| P20-ATG2F (5′ACGTATGGATCCGGCTTTGTCTACGATGAAC3′); | (SEQ ID NO: 14) |
| P20-ATG3F (5′ACGTATGGATCCGAACAAGCCCGGGCCGTTT3′); | (SEQ ID NO: 56) |
| or | |
| P20-pTrcR (5′ACGCATGAATTCTGTTCTGAGTTCCCGCT3′). | (SEQ ID NO: 15) |

These primers place unique BamH1 and EcoR1 restriction sites on the five and three prime sides of the NcP20 ORF, respectively. The PCR products obtained were checked on a 1% agarose gel for size and purified using a Qiaquick PCR purification kit. DNA from the purified PCR product and pTrcHisB vector (Invitrogen) were then digested with both Bard and EcoR1 restriction enzymes for three hours at 37° C. The digested DNA were purified using a Qiaquick column and checked on a 1% agarose gel. The three PCR products of NcP20 were then ligated separately into the pTrcHisB vector and transformed into E. coli DH5α. Individual recombinants were screened for inserts by PCR using primers pTrcHisFwd (5′GAGGTATATATTAATGTATCG3′) (SEQ ID NO: 50) and P20-pTrck The sequence of the constructs made were confirmed by cycle sequencing. This strategy ensured the initiation codon of NcP20 was cloned in-frame into the pTrcHisB vector, which following transcription and translation should produce a polypeptide. Subsequently, E. coli containing recombinant DNA were grown in LB medium containing ampicillin and at mid-log phase were induced with 1 mM IPTG. After several hours, the bacteria were collected by centrifugation and solubilised in guanidinium lysis buffer. His-tagged protein was purified using Ni-NTA (Qiagen) resin following the manufacturers instructions for preparation of denatured E. coli cell lysate. Proteins were analysed on 14% SDS-PAGE gels by starting with Coomassie blue.

Identification of Native NcP20 Antigen Fragments

One microgram of recombinant fragment of NcP20 purified from E. coli, was injected subcutaneously into five, 9 week-old QS mice with Freund's complete or Freund's incomplete adjuvant 4 weeks apart. Mice were bled from the tail vein 3 weeks after the boost. Sera were pooled and used in immunoblotting against reduced tachyzoite antigen.

Vaccination Trial

Ten groups of 9 mice were injected twice, subcutaneously in the scruff of the neck, 4 weeks apart, with one of the following treatments (ANZCCART 1998, ISBN 0 646 24923 1):

Group 1: 0.1 ml Freund's complete adjuvant (FCA) followed by a boost with 0.1 ml Freund's incomplete adjuvant (FIA) only;

Group 2: 0.1 ml FCA plus 1 μg NcP20;

Group 3: 0.1 ml FIA only;

Group 4: 0.1 ml FIA plus 1 μg NcP20;

Group 5: 0.1 ml FIA plus 25 μg glucosaminylmuramyl dipeptide (GAP),

Group 6: 0.1 ml FIA plus 25 μg GMDP plus 1 μg NcP20;

Group 7: 0.1 ml 0.9% NaCl containing 10 μg Quil A only;

Group 8: 0.1 μl 0.90% NaCl containing 10 μg Quil A plus 1 μg NcP20;

Group 9: saline ((+)ve control)

Group 10: no treatment (−ve control).

Mice (all groups except group 10) were then challenged three weeks after the second injection with 7.5×10⁵ culture-derived tachyzoites of NC-Liverpool subcutaneously. Changes in mean group body weight (GW) between 14-27 days post infection (DPI) with N. caninum were determined and analysed by a one-factor-repeated measures analysis of variance, with treatment as the factor and time as the repeated measure. All the sampling times were included in the analysis. Two mice (one in group 1 and one in group 6) were removed from the analysis because their changes in body weight were anomalous. The details of the differences among treatments were assessed using a posteriori Tukey HSD multiple comparison test.

Enzyme-Linked Immunosorbent Assay Using the NcP20 Fragment

Histidine-tagged; recombinant NcP20 fragment (purified on Ni-NTA resin as described previously) was coated onto 96-well microtitre plates at 1 μg/well diluted in ELISA buffer 1 (70 mM NaHCO₃, 29 mM Na₂CO₃, 3.1 mM NaN₃, pH 9.6). Following overnight incubation at 4° C., the plates were washed 3 times in wash buffer (PBS, 0.03% Tween 20, pH 7.2). Serum samples were diluted 1:25 using ELISA buffer 2 (0.5 g bovine haemoglobin, 0.3% Tween 20, 3.1 mM NaN₃, pH 7.2 in PBS), and 100 μl of each sample was added in duplicate. The plates were incubated overnight at 4° C. and then washed as before. One hundred μl of biotinylated antibody to mouse IgG (The Binding Site, UK) was added to each well at a dilution of 1:6000 in ELISA buffer 2. Following a 1 hour incubation at 37° C. and washing, each well was coated with 100 μl of ExtrAvidin alkaline phosphatase (Sigma, USA) at a dilution of 1:5000 in ELISA buffer 2. After incubation for 1 hour at 37° C. the plates were again washed and 100 μl of Alkaline Phosphatase Substrate 104 (Sigma, USA) was added at a concentration of 1 mg/ml in ELISA buffer 3 (58 mM NaHCO₃, 42 mM Na₂CO₃, 2 mM MgCl_{2.6}H₂O, pH 98). The plates were incubated at 37° C. for 30 min, allowing sufficient colour development. The absorbance reading of each well at 405 nm was determined using an electronic plate reader (Biorad).

mRNA Sequence of NcP20

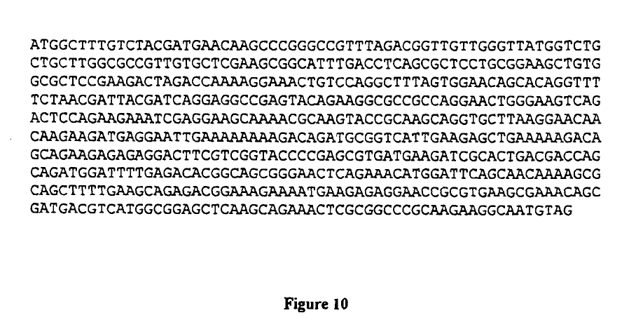

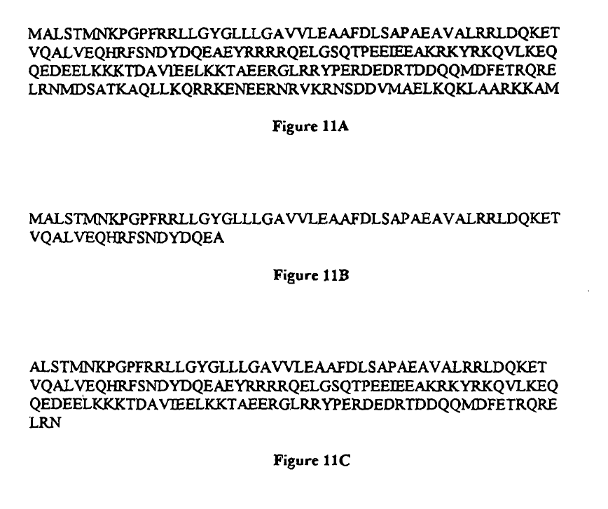

Initial sequencing suggested a short reading frame encoding a small polypeptide of approx. 12 kDa was encoded by NcP20 (see above); however further sequencing of these mRNAs resolved anomalous bases extended the reading frame in the 3′ direction. The cDNA coding sequence, along with the predicted protein sequence of NcP20 are provided in FIGS. 10 and 11A (SEQ ID NO's: 9 and 3 respectively). The coding region predicts a protein translation product of 23.4 kDa with a pI of 9.78.

Production of Full-Length NcP20

NcP20 was re-cloned into the pET25b((+)) vector and full-length NcP20 recombinant protein produced. Complimentary DNA synthesized from N. caninum tachyzoite mRNA was amplified by PCR with primers p30ATG2F (5′ACGTATGGATCCGGCTTTGTCTACGATGAAC3′) (SEQ ID NO; 14) and pET25p20R4 (5′ACGTATAAGCTTTGCCTTCTTGCGGGCCGCGA3′) (SEQ ID NO: 57). The PCR product obtained was digested with BamH1 and Hind111, and directly cloned into pET25b((+)) vector also digested with these enzymes. Escherichia coli transformants were screened by PCR with vector-based primers T7P (5′TTAATACGACTCACTATAGGG3′) (SEQ ID NO: 53) and I7T (5′GCTAGTTATTGCTCAGCG3′) (SEQ ID NO; 54) in order to identify clones with inserts. These inserts were purified and sequenced by cycle sequencing from a number of clones in order to confirm the correct sequence and reading frame had been cloned into the vector. A bacterial clone containing NcP20 cloned correctly into pET25b((+)) was chosen for further expression studies. Expression of NcP20 from pET25b((+)) results in the expression of a protein predicted to have a molecular weight of 30 kDa (including the His tag fusion).

Recombinant NcP20 protein was purified from a 50 ml bacterial culture grown in L-broth containing ampicillin The bacteria were pelleted by centrifugation, and the pellet was resuspended in lysis buffer containing lysozyme and incubated on ice for 30 min. The bacterial cells were then disrupted by sonication, and the sonicate passed through a 19.5 gauge needle. The extract was then centrifuged at 10,000 g to clear. His tagged NcP20 was then purified from the extract by Ni-NTA resin chromatography using recommendations, where possible, from the manufacturer. Contaminating proteins were removed by washing the column with 8 washes of 50 mM Na₂PO₄, pH 8.0, 300 mm NaCl 20 mM imidazole, after which the NcP20 was eluted with 2 washes of 50 mM NaH₇₂PO₄, pH 8.0, 300 mm NaCl, 50 mM imidazole. Elutions were checked by SDS-PAGE and dialysed against 0.9% saline. Protein solutions were lyophilized for long term storage.

Results

Identification of Antigens for APAb Preparation

Immunoblotting of sera from resistant (vaccinated) and acutely infected naive Balb/C mice to antigen from NC-Liverpool and NC-SweB1 were studied. A group of antigens were identified by blotting using sera from resistant mice in the range 15-30 kDa which were absent or significantly less immunogenic in tracks probed with sera from acutely infected naive mice. An APAb was therefore prepared from antigens over this specific size range. The specificity of the APAb to N. caninum antigen was investigated by immunoblotting. Immunoreactive bands of approx. 17-18 kDa were faintly detected under non-reducing and reducing conditions respectively.

Gene Isolation and Characterisation

Four positive clones were isolated from the cDNA library by immunoscreening with the APAb. PCR products were obtained from the cDNA clones with sizes of approximately 1200, 1000, 900 and 800 bp. Sequence from the 1200 bp clone was homologous to NCGRA7/NCDG1 (Lally et al. 1996, Lally et al. 1997) and not studied further. Sequence from the 1000 bp clone predicted limited protein sequence homology of the gene product with the Gra1 protein of T. gondii (Cesbron-Delauw et al. 1989) which was detected by a TblastX search of the GenBank database. Hence this gene was called NCGRA1 (GenBank accession number AF199030) and is described elsewhere.

The remaining two cDNA sequences isolated from the cDNA library were homologous to each other. Eight additional cDNA sequences (ESTs) homologous to these cDNAs, were also identified amongst a small EST database maintained at UTS and a consensus DNA sequence was derived from all of them extending the data to approximately 1088 bp. The final cDNA consensus was 280 bases longer at the 5′ end than the original two cDNAs isolated and possessed three potential initiation codons at positions 1, 160 and 175.

NcP20/NcP20 clustered in the ToxoDB with Ctoxqual_{—}1252, a cluster containing 22 ESTs of T. gondii. Of potential significance is that the two most similar T. gondii sequences (with the highest probability of a match) were ESTs derived from a bradyzoite cDNA (TgEST zz70do9.rl and TgESTzz46do3.rl). Thus it was concluded that a T. gondii homologue of NcP20 exists.

Production and Immunisation of a Fragment NcP20 in E. coli

Identification of a Fragment of Native Tachyzoite Antigen Encoded by NcP20

cDNA sequence analysis predicted the presence of three potential start codons in NcP20. Consequently, primers were designed to PCR amplify the ORF from each of these potential start codons and the products were cloned individually, in-frame into pTRcHis. E. coli containing PCR product derived from primers P20-ATG3F and P20-pTrcR, encoding the shortest ORF (protein fragment encoded by this ORF provided as SEQ ID NO: 6), expressed a protein with a mobility of approximately 30 kDa which could be purified from bacterial extracts by chromatography. No recombinant protein was obtained from E. coli containing the other two constructs. Injection of purified protein into QS mice generated IgG antibodies which detected, by immunoblotting, a 20 kDa tachyzoite antigen in NC-SweB1. No antigen was detected by this pooled sera in extracts of NC-Liverpool.

Vaccination Experiment

The statistical analysis was a one-factor analysis of variance, with the various treatments as the factor. The data analysed were the change in mouse weight through time, calculated using a separate linear regression for each mouse (FIG. 12). Only mice with data for all of the days were used in the analysis. The significant factor (P<0.001) indicated that the mice change weight differently among some of the groups. A multiple comparison test showed that groups 1 (FCA) and 2 (FCA(+)NcP20) were not different from 10 (the negative control) in that they maintained their body weight close to their starting weight. Mice in all the other groups behaved like those in group 9 (the positive control) in that they lost weight over the course of the experiment. Thus it was concluded that injection of mice with either FCA with or without recombinant NcP20 provided complete protection against weight loss that normally occurs during an acute infection of N. caninum. All mice infected with N. caninum showed clinical signs associated with the infection, which were a ruffled coat from day 14 post infection

Enzyme-Linked Immunosorbent Assay Using NcP20

FIG. 13 shows the results obtained by ELISA using recombinant NcP20 fragment and three test sera from mice. The negative control serum is derived from a pregnant QS mouse injected with saline at day 8 of gestation whereas the other 2 sera (labeled positive sera) are from pregnant QS mice injected subcutaneously at day 8 of gestation with 1 million tachyzoites of NC-Liverpool. All mice were bled from the heart on day 21 of gestation and sera prepared by standard procedures. The antibody response to NcP20 fragment in the pregnant mice receiving NC-Liverpool, as determined from the ELISA OD readings, was significantly higher than that found in the pregnant mouse receiving saline, thereby indicating the ELISA detected IgG antibodies in those mice infected with N. caninum.

Full-Length NcP20

Two of the three potential ORFs described above encode fragments of NcP20, whilst the third encodes full-length NcP20. The inability of two of the expression constructs (including the one encoding the full-length protein) to produce recombinant protein was found to be the result of problems with the expression system and not that the ORFs did not encode fragments of NcP20.

Full-length NcP20 (FIG. 11A) was re-cloned into the pET25b((+)) vector and recombinant protein produced and purified. This protein was used in the experiments described in Example 4.

Discussion

The isolation and characterisation of a new gene from N. caninum, called NcP20 is reported The gene was isolated by modifying a strategic immunoscreening technique. The immunoscreening strategy used was devised in order to avoid isolating antigen sequences that are cross-reactive to the dense granule antigen NCDG1 of N. caninum which is highly immunogenic. Consequently, antigens blotted onto PVDF membranes that were smaller than 30 kDa were used to select antibody from pooled sera of mice made resistant to a lethal challenge of NC-Liverpool by previous infection with NC-SweB1. The selected antibody was then used to immunoscreen the cDNA expression library.

Two genes, previously undescribed from N. caninum were isolated by immunoscreening, one of which was homologous to the GRA1 gene of T. gondii which is described elsewhere. GRA1 was the first dense granule antigen of T. gondii cloned because it formed a major reactive component of the excretory/secretory fraction from T. gondii (Darcy et al. 1988, Cesbron-Delauw et al. 1989). This fraction contains antigens which induce a protective antibody response and thus prompted the isolation and characterisation of many dense granule antigens from T. gondii to date. Homologues of four of the genes coding for these antigens have now been described for N. caninum: NCGRA1, NCGRA2, NCGRA6 and NCGRA7.

GRA1 is released from bradyzoites of T. gondii and so is thought to be a marker of chronic infection in toxoplasmosis. In N. caninum, a similar function may also apply to NcGra1 and NcP20 since the sera used for the isolation of these clones was derived from mice made resistant to an acute infection of NC-Liverpool by prior infection with NC-SweB1.

A homologue of NcP20 was detected in T. gondii by searching the GenBank database with the NcP20 sequence. Other parasite taxa, such as Neospora hughesi, Hammondia heydorni and Hammondia hammondi are therefore also expected to contain homologues of NcP20, because these taxa are very closely related to N. caninum. As the present inventors have shown that the NcP20 protein is suitable to raise high titres of protective antibodies in mice, it will be expected that the protein will act similarly in other animals upon immunisation.

The high prevalence of N. caninum in cattle along with its high rate of congenital transmission suggests the development of vaccines are warranted. Prior evidence suggests a role for low molecular weight antigens during the induction of cellular immunity in neosporosis. Antigens in the 15-30 kDa range induce proliferation of CD4⁽⁺⁾ cells thereby stimulating increased production of gamma interferon which in turn suppresses the growth of N. caninum tachyzoites and encourages resistance against N. caninum. These low molecular weight antigens also appear more immunogenic in immune mice and outbred (QS strain) mice resistant to clinical disease. Thus low molecular weight antigens such as NcP20 may well have a role in inducing cell mediated immunity against N. caninum.

Recombinant NcP20, purified from E. coli, was evaluated in a variety of vaccine formulations for its ability to protect susceptible mice against an acute infection of N. caninum. Freund's complete adjuvant, with or without recombinant NcP20, was able to provide complete protection against weight loss associated with the acute infection of N. caninum.

In summary, a new gene of N. caninum have been isolated and characterised, called NcP20. Its isolation is significant because this is the first report of using sera, from animals shown to be resistant to an acute infection with N. caninum, for immunoscreening of cDNA expression libraries.

### Example 4

Materials and Methods

Prevention of Transplacental Transmission Using a Mouse Model

A Neospora caninum lysate was prepared as follows. N. caninum tachyzoites were collected as described above and stored as dry pellets at −20° C. until required. Pellets were resuspended in lysis buffer (20 mM Tris Cl pH 7.5, 0.15M NaCl, 1% Triton X-100, 1 mM Ethylenediaminetetraacetic acid, 1 mM Benzamidine, 1 mM Phenymethylsulphonyl fluoride and 2 mM Dithiothreitol) and incubated on ice for 1 hr. The parasites were then sonicated 3 times at 50W/20 KHz for 15 sec and spun at 3000 g for 10 min to remove insoluble debris. The lysate was dialysed overnight at 4° C. against PBS and the concentration was determined using the Lowry protein assay (Biorad).

120 female Qs mice at 4 weeks of age, were divided into 6 groups as follows:

Group 1—challenge only

Group 2—VSA-3 adjuvant only

Group 3—N. caninum lysate (10 μg/mouse)

Group 4—NcP20 (10 μg/mouse)

Group 5—p24B (10 μmouse)

Group 6—NcP20(+)p24B (10 μg of each/mouse)

All injections were delivered subcutaneously in a volume of 150 μl. Group 1 received injections of saline only. Groups 3-6 had their formulation delivered with VSA-3 adjuvant, which comprised ⅓ of the total vaccine volume. Four weeks later, booster injections were given, identical to those described above.

Mating and Pregnancy

Mice were housed individually, overnight, with a male Qs mouse following synchronisation of ovulation using Folligon (Pregnant Mare Serum Gonadotrophin) and Chorulon (human Chorionic Gonadotrophin. Female mice were inspected for the presence of a vaginal mucoid plug and the plugged females (potentially pregnant) were separated from the non-plugged females.

Four weeks following the booster injection (day 5 of gestation), plugged mice in all groups received a challenge of NC-Liverpool tachyzoites. The dose of 10⁶ parasites were delivered by subcutaneous injection.

Non-plugged mice were euthanased and serum was collected. This was to provide an estimate of antibody levels at the time of challenge in plugged mice.

Plugged, challenged mice were housed individually and allowed to carry their pregnancy to term. Mice were checked daily until all had given birth. Seven days after giving birth, dams and surviving pups were euthanased. Pup brains were removed and snap-frozen in liquid nitrogen, prior to being transferred to −20° C. for short-term storage. Serum was collected from the dams for analysis of antibody levels.

DNA Extraction and PCR

Individual pup brains were homogenised in 3 ml of DNAzol. Proteinase K was added to a final concentration of 100 μg/ml and tubes were left at room temperature until lysis was complete (i.e. no undigested tissue visible). 25 ml of 100% ethanol was added and the tube inverted until DNA precipitation was complete. DNA precipitate was transferred to a sterile tube and washed twice with 70% DNAzol/30% ethanol and then once with 75% ethanol. All liquid was pipetted off and the DNA pellet was resuspended in 750 μl sterile water. Extracted DNA was stored at 4° C. until PCR was performed.

All PCR preparation was carried out in a Class 2 Biological Safety Cabinet using aerosol barrier pipette tips. A distilled water negative control was prepared with every set of PCR reactions and positive controls of N. caninum genomic DNA were also included.

Parasite DNA was detected using the primers CR3 (5′-ATATACTACTCCCTGTGAGTT-3′) (SEQ ID NO: 58) and CR4 (5′-GTAATCTGAAAGCGAATAGAG-3′) (SEQ ID NO: 59), designed to amplify a 300 bp fragment of the ITS-1 region of the 18S ribosomal DNA from N. caninum. The PCR reaction mixture (25 μl) consisted of 1×PCR Buffer, 2.5 mM MgCl₂, 0.2 mM each dNTP, 1.1U of Taq polymerase and 0.25 μM of each primer. 2.5 μl of the extracted DNA was used in each PCR reaction. Thermal cycling conditions were: 95° C. for 2 min; 35 cycles of 95° C. for 45 sec, S0° C. for 45 sec. 72° C. for 1.5 min; 72° C. for 5 min. Following amplification, 5 μl aliquots of each reaction were electrophoresed on a 2% agarose gel along with a 100 bp marker, stained with ethidium bromide and viewed using a UV transilluminator. Transmission was determined by the presence of a band of appropriate size in the PCR reaction. The numbers of positive and negative pups were tallied for each litter and each group. Chi square analysis was used to determine if transmission rates among groups were significantly different than the saline vaccinated challenge only mice.

Detection of anti-N. caninum Antibodies

The level of IgG, IgG1 and IgG2a antibodies specific to N. caninum in the serum from the adult mice was measured by ELISA. ELISA plates were coated with N. caninum lysate (batch used for immunization), NcP20 antigen or 24B antigen diluted at a concentration of 10 μg/ml in carbonate buffer (70 mM NaHCO₃, 29 mM Na₂CO₃, 3.1 mM NaN₃, pH 9.6) and incubated at 4° C. overnight. Plates were washed 3 times with PBS/0.03% Tween (PBST) and serum diluted 1:100 in blocking buffer (0.05% bovine haemoglobin, 0.3% Tween, 3.1 mM NaN₃ in PBS, pH 7.2) was added to each well in duplicate. Plates were again incubated overnight at 4° C. and then washed three times with PBST. Anti-mouse IgG-Alkaline Phosphatase, anti-mouse IgG1-Biotin and anti-mouse IgG2α-Biotin antibodies were diluted 1:6000 in blocking buffer, added to wells and plates were incubated overnight at 4° C. Plates incubated with IgG1 and IgG2a were washed 3 times with PBST, and Extavidin-Alkaline Phosphatase at a dilution of 1:5000 in blocking buffer was added to the wells. These plates were incubated at 37° C. for 1 hr. All plates were washed 3 times with PBST. ρ-nitrophenylphosphate at a concentration of 1 mg/ml in developing buffer (58 mM NaHCO₃, 42 mm Na₂CO₃, 2 mM MgCl₂.6H₂O, pH 9.8) was added to the wells. Plates were incubated at 37° C. for 15 min and read in an electronic plate reader at an absorbance of 405 ml. Differences in absorbance between groups was analysed by ANOVA, followed by a Tukey-Kramer multiple comparison test.

Results and Discussion

Overall, the mean number of mice that were plugged following overnight mating was 68% Plugging success rates within each group were variable, however, ranging from a low of 50% for the NcP20(+)24B group to a high of 88% for the challenge only group. DNA was extracted from 684 pups which survived to 7 days of age. PCR was done at least once on all samples extracted and all samples that gave a faint positive result were repeated. The number of positive and negative PCR results was calculated for each litter and for each group. The rate of transmission in mice injected with saline (the positive control) was 76% (that is 76% of the pups in this group contained detectable levels of N. caninum DNA). Significant reductions in parasite transmission were observed in some treatment groups. Those vaccinated with N. caninum lysate showed transmission in 62.5% of the pups, a reduction compared to the controls of 17.8%; (P=0.0485). Both groups individually “vaccinated” with recombinant antigens (p24B or NcP20) transmitted N. caninum to 66% (NcP20) and 70% (p24b) of offspring, a reduction of 13.2 and 7.8% respectively. However in the group given the two recombinant proteins together (NcP20(+)p24B) 51% of offspring only were positive, a reduction of transmission of 32.9%; (P=0.0009).

ELISA's were performed using NcP20 or 24B as the coating antigen, to estimate the specific antibody response to these recombinant proteins by mice during the course of the experiment. Mice vaccinated with NcP20(+)24B had a significantly greater NcP20 specific antibody response than mice vaccinated with saline (P<0.05) and both mice vaccinated with 24B alone and NcP20(+)24B had a significant 24B specific antibody response, compared with challenge only mice (P<0.05). NcP20 vaccinated mice did not have a significant NcP20 specific antibody response compared with saline vaccinated mice and the response in this group was significantly less than the response in mice vaccinated with NcP20(+)24B (P<0.05).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Atkinson, R. A., Harper, P. A. W., Ryce, C., Morrison, D. A. and Ellis, J. T. (1999) Parasitolog 118:363-371.

Barber, J. S., Holmdahl, O. J. M., Owen, M. R., Guy, F., Uggla, A. and Trees, A. J. (1995) Parasitology 111:563-568.

Brennan, F R., Bellaby, T., Helliwell, S. M., Jones, T D., Kamstrup, S., Dalsgaard, K., Flock, J -I. and Hamilton, W. D. O. (1999) Journal of Virology 73:930-938.

Cardoso, A. I., Blixenkrone-Moller, M., Fayolle, J., Liu, M, Buckland, R. and Wild, T. F. (1996) Virology 225:293-299.

Cesbron-Delauw, M. F. et al. (1989) Proceedings of the National Academy of Sciences of the United States of America 86:7537-7541.

Chakrabarti, P. and Chakrabarti, S. (1998) Journal of Molecular Biology 284:867-873.

Chou, P. T. and Fasman, G. D. (1973) Journal of Molecular Biology 74.263-281.

Cohen, A. D., Boyer, J D. and Weiner, D. B. (1998) FASEB J. 12:1611-1626.

Cunningham, B. C. and Wells, J. A. Science (1989) 244:1081-1085.

Darcy, F. et al. (1988) Parasite Immunology 10553-567

Deleage, G. and Roux, B. (1987) Protein Engineering 1289-294.

Dubey, J. P, Carpenter, J. L., Speer, C. A., Topper, M. J. and Uggla, A. (1988) Journal of the American Veterinary and Medical Association 192:1269-1285.

Eisenbraun, M. D., Fuller, D. H. and Haynes, J. R. (1993) DNA and Cell Biology 12:791-797.

Ellis, J. T. et al. (2000) Parasitology 120:383-390.

Frishman, D. and Argos, P. (1996) Protein Engineering 9:133-142.

Fynan, E. F., Webster, R. G., Fuller, D. H., Haynes, J. R., Santoro, J. C. and Robinson, H. L. (1993) Proceedings of the National Academy of Sciences of the United States of America 90:11478-11482.

Garnier, J, Osguthorpe, D J. and Robson, B. (1978) Journal of Molecular Biology 120:97-120.

Garnier, J., Gibrat, J -F and Robson, B. (1996) Methods in Enzymology 266.540-553.

Geourjon, C. and Deleage, G. (1994) Protein Engineering 7:157-164.

Geourjon, C. and Deleage, G. (1995) Computer and Applied Bioscience 11:68-684.

Gibrat, J. F. Garnier, J. and Robson, B. (1987) Journal of Molecular Biology 198:425-443.

Hemphill, A. et al. (1997b) Parasitology 115:371-380.

Hood, E. E. and Jilka, J. M. (1999) Current Opinions in Biotechnology 10:382-386

Howe, D. K., Mercier, C., Messina, M. and Sibley, L. D (1997) Molecular and Biochemical Parasitology 86:29-36.

Howe, D. K. and Sibley, L. D. (1997) Methods 13:123-133.

Kapustra J., Modelska A., Figlerowicz M., Pniewski T., Letellier M., Lisowa O., Yusibov V., Koprowski H, Plucienniczak A. and Legocki A. B. (1999) FASEB J 13:1796-1799.

Kissinger, J. C., Gajria, B., Li, L., Paulsen I. T. and Roos D. S. (2003) Nucleic Acids Research 31:234-236.

Knoll, L. J., Furie, G. L. and Boothroyd, J C. (2001) Molecular and Biochemical Parasitology 116(1):11-16

Lally, N. C., Jenkins, M. C. and Dubey, J. P (1996) Clinical and Diagnostic Laboratory Immunology 3:275-279.

Lally, N. et al. (1997) Molecular and Biochemical Parasitology 87239-243.

Levin, J., Robson, B. and Garnier, J (1986) FEBS 205:303-308.

Levin, J. (1997) Protein Engineering 7:771-776.

Mason, H. S., Ball, J. M., Shi, J. J., Jiang, X., Estes, M. K. and Arntzen, C. J. (1996) Proceedings of the National Academy of Sciences of the United States of America 93:5335-5340.

McAllister, M. M., Dubey, J. P., Lindsay, D S., Jolley, W. R., Wills, R. A. and McGuire, A. M. (1998) International Journal of Parasitology 28 1473-1478.

Mehta, P K., Heringa, J. and Argos, P. (1995) Protein Science 4.2517-2525.

Mercier, C., Lecordier, L., Darcy, D., Deslee, A., Murray, B., Tourvielle, P., Maes, A., Capron, A. and Cesbron-Delauw, M. F (1993) Molecular and Biochemical Parasitology 58:71-82.

Modelska, A., Dietzschold, B., Sleysh, N., Fu, Z. F., Steplewski, K., Hooper, D. C., Koprowski, H. and Yusibov, V. (1998) Proceedings of the National Academy of Sciences of the United States of America 95:2481-2485.

Montgomery, D. L, Shiver, J. W., Leander, K. R., Perry, H. C., Friedman, A., Martinez, D., Ulmer, J. B., Donnelly, J J. and Lui, M. A. (1993) DNA and Cell Biology 12:777-783.

Needleman, S. B. and Wunsch, C. D. (1970) Molecular Biology 48. 443-453.

Prince, J. B., Araujo, F. G., Remington, J. S., Burg, J. L., Boothroyd, J C. and Sharma, S. D. (1989) Molecular and Biochemical Parasitology 34:3-14.

Rost, B. and Sander, C. (1993) Journal of Molecular Biology 232:584-599.

Rost, B and Sander, C. (1994) Proteins 20:216-226.

Rost, B. (1996) Methods in Enzymology 266:525-539.

Salamov, A. A. and Solovyev, V. V. (1995) Journal of Molecular Biology 247:11-15.

Sedegah, M., Hedstrom, R, Hobart, P. and Hoffman, S L (1994) Proceedings of the National Academy of Sciences of the United States of America 91:9866-9870.

Sibley, L. D., Mordue, D. G., Su, C. L., Robben, P. M. and Howe, D. K. (2002). Phil. Trans. Roy. Soc. Lond. Series B: Biol. Sci. 357:81-88.

Soldati, D., Kim, K., Kampmeier, J., Dubremetz, I -F. and Boothroyd, J. C. (1995) Molecular and Biochemical Parasitology 74:87-97

Stenlund, S., Björkman, C., Holmdahl, O. J. M., Kindahl, H. and Uggla, A. (1997) Parasitology Research 83 214-219.

Stultz, C. M., White, J. V. and Smith, T. F. (1993) Protein Science 2:305-314.

von Heijne, G. (1986) Nucleic Acids Research 14.4683-4690

Wang, B., Ugen, K. E., Srikantan, V., Agadjanyan, M. G., Dang, K., Refaeli, Y., Sato, A. I., Boyer, J., Williams, W V. and Weiner, D. B. (1993) Proceedings of the National Academy of Sciences of the United States of America 90.4156-4160.

Xiang, Z. Q., Spitalnik, S., Tran, M., Wunner, W H., Cheng, J. and Ertl, H. C. (1994) Virology 199:132-140.

Yang K., Mustafa F., Valsamakis A., Santoro J. C., Griffin D. E. and Robinson H. L. (1997) Vaccine 15:888-891.

1. A substantially purified polypeptide comprising an amino acid sequence selected from the group consisting of:
a) an amino acid sequence as set forth in SEQ ID NO:1,
b) an amino acid sequence as set forth in SEQ ED NO:4,
c) an amino acid sequence which is at least 95% identical to SEQ ID NO:1, and
d) an immunogenic fragment of any one of a) to c),
wherein the polypeptide raises an immune response against N. caninum when administered to an animal.

2. The polypeptide of claim 1 which comprises an amino acid sequence as set forth in SEQ ID NO: 1.

3. The polypeptide of claim 1 which comprises an amino acid sequence as set forth in SEQ ID NO:4.

4. A composition comprising a pharmaceutically acceptable carrier and a polypeptide according to claim 1.

5. The composition of claim 4 which comprises an additional polypeptide which raises an immune response against N. caninum when administered to an animal.

6. The composition of claim 5, wherein the additional polypeptide comprises an amino acid sequence selected from the group consisting of
a) an amino acid sequence as set forth in SEQ ID NO:2,
b) an amino acid sequence as set forth in SEQ ID NO:5,
c) an amino acid sequence as set forth in SEQ ID NO:6,
d) an amino acid sequence which is at least 95% identical to SEQ ID NO:2, and
e) an immunogenic fragment of any one of a) to d).

7. The composition of claim 4 further comprising an adjuvant.

8. The composition of claim 7, wherein the adjuvant is selected from the group consisting of aluminum salts, water-in-soil emulsions, oil-in-water emulsions, saponin, QuilA and derivatives, iscoms, liposomes, cytokines, DNA, microencapsulation in a solid or semi-solid particle, Freunds complete and incomplete adjuvant or active ingredients thereof, DEAE dextran/mineral oil, Alhydrogel, Auspharm adjuvant, and Algammulin.

9. A method of raising an immune response against N. caninum in an animal, the method comprising administering to the animal an effective amount of a composition according to claim 4, to produce an immune response against the polypeptide thereby providing an immune response against N. caninum.

10. The method according to claim 9, wherein administering of the composition is by injection via intramuscular, subcutaneous, intradermal or intraaperitoneal routes, or included as an additive in feed or water.


SEQUENCE LISTING (60 sequences)

SEQ ID NO: 1 - protein, 242 aa, Neospora caninum
MDPKVESQTNVPSGAEAEQPKAGEAQATVENGNTSAPDAQVKSQASSEDVVAQSSEDFSGKLQANSGIVSFGDSAAGSGAFNSMDVQNFLQRYATSKMFGVPPHFFQSRESLRVWGADHLTDPMVQPYEKDDQNLPNPFHVSLPGYSPSLCKYVLTKGEKPPRDPLLGPEITIYPPTWIPHWEPDPNFKPQAYNFNWEENGTFQMERLPYAKAVFDPADGSAHGMYKQAYPYTAYPYGVPRV

SEQ ID NO: 2 - protein, 198 aa, Neospora caninum
MALSTMNKPGPFRRLLGYGLLLGAVVLEAAFDLSAPAEAVALRRLDQKETVQALVEQHRFSNDYDQEAEYRRRRQELGSQTPEEIEEAKRKYRKQVLKEQQEDEELKKKTDAVIEELKKTAEERGLRRYPERDEDRTDDQQMDFETRQRELRNMDSATKAQLLKQRRKENEERNRVKRNSDDVMAELKQKLAARKKAM

SEQ ID NO: 3 - protein, 211 aa, Neospora caninum
MFTGKRWILVVAVGALVGASVKAADFSGRGTVNGQPVGSGYSGYPRGDDVRESMAAPEDLPGERQPETPTAEAVKQAAAKAYRLLKQFTAKVGQETENAYYHVKKATMKGFDVAKDQSYKGYLAVRKATAKGLQSAGKSLELKESAPTGTTTAAPTEKVPPSGPRSGEVQRTRKEQNDVQQTAEMLAEEILEAGLKKDDGEGRGTPEAEVN

SEQ ID NO: 4 - protein, 159 aa, Neospora caninum
MDVQNFLQRYATSKMFGVPPHFFQSRESLRVWGADHLTDPMVQPYEKDDQNLPNPFHVSLPGYSPSLCKYVLTKGEKPPRDPLLGPEITIYPPTWIPHWEPDPNFKPQAYNFNWEENGTFQMERLPYAKAVFDPADGSAHGMYKQAYPYTAYPYGVPRV

SEQ ID NO: 5 - protein, 68 aa, Neospora caninum
MALSTMNKPGPFRRLLGYGLLLGAVVLEAAFDLSAPAEAVALRRLDQKETVQALVEQHRFSNDYDQEA

SEQ ID NO: 6 - protein, 152 aa, Neospora caninum
ALSTMNKPGPFRRLLGYGLLLGAVVLEAAFDLSAPAEAVALRRLDQKETVQALVEQHRFSNDYDQEAEYRRRRQELGSQTPEEIEEAKRKYRKQVLKEQQEDEELKKKTDAVIEELKKTAEERGLRRYPERDEDRTDDQQMDFETRQRELRN

SEQ ID NO: 7 - DNA, 729 bp, Neospora caninum
atggatcctaaagtggagagtcaaacaaatgtgccatctggcgcagaggcagagcagcccaaggcaggagaggcacaagcaactgtggagaacggtaatacttcagctccggatgctcaggtgaagtcccaagcgtcctccgaagatgtggtagcgcagtcgtcagaagacttcagcggaaagcttcaggccaactcaggcattgtgagcttcggagactctgctgctggaagtggtgcgttcaacagtatggacgtgcagaactttctccagcgttacgcaacgagcaagatgtttggagttccgccgcatttcttccaaagcagagaaagcctccgagtctggggagctgaccacctcaccgatcccatggtgcagccttacgagaaagacgatcagaacctacccaatccctttcatgtttcgctacctgggtactctccgtctctctgcaagtacgttctgaccaagggcgagaagcctccccgcgatcccctcctcggacctgagattaccatttacccgcctacgtggattccgcactgggaacccgatcccaatttcaagccacaggcttacaatttcaactgggaggagaacggcacatttcagatggaacggttgccgtacgcgaaagcggtcttcgatccagcagacggctcagcacacggcatgtacaagcaagcctacccttacacagcgtatccatacggtgttccgcgcgtctag

SEQ ID NO: 8 - DNA, 597 bp, Neospora caninum
atggctttgtctacgatgaacaagcccgggccgtttagacggttgttgggttatggtctgctgcttggcgccgttgtgctcgaagcggcatttgacctcagcgctcctgcggaagctgtggcgctccgaagactagaccaaaaggaaactgtccaggctttagtggaacagcacaggttttctaacgattacgatcaggaggccgagtacagaaggcgccgccaggaactgggaagtcagactccagaagaaatcgaggaagcaaaacgcaagtaccgcaagcaggtgcttaaggaacaacaagaagatgaggaattgaaaaaaaagacagatgcggtcattgaagagctgaaaaagacagcagaagagagaggacttcgtcggtaccccgagcgtgatgaagatcgcactgacgaccagcagatggattttgagacacggcagcgggaactcagaaacatggattcagcaacaaaagcgcagcttttgaagcagagacggaaagaaaatgaagagaggaaccgcgtgaagcgaaacagcgatgacgtcatggcggagctcaagcagaaactcgcggcccgcaagaaggcaatgtag

SEQ ID NO: 9 - DNA, 636 bp, Neospora caninum
atgttcacggggaaacgttggatacttgttgttgccgttggcgccctggtcggcgcctcggtaaaggcagccgatttttctggcaggggaaccgtcaatggacagccggttggcagcggttattccggatatccccgtggcgatgatgttagagaatcaatggctgcacccgaagatctgccaggcgagaggcaaccggagacacccacggcggaagctgtaaaacaggcagcggcaaaagcttatcgattactcaagcagtttactgcgaaggtcggacaggaaactgagaacgcctactaccacgtgaagaaagcgacaatgaaaggctttgacgttgcaaaagaccagtcgtataagggctacttggccgtcaggaaagccacagctaagggcctgcagagcgctggcaagagccttgagcttaaagagtcggcaccgacaggcactacgactgcggcgccgactgaaaaagtgccccccagtggcccgtgatcaggtgaagttcaacgtactcgtaaggagcaaaatgacgtgcagcaaaccgcagagatgttggctgaggaaattcttgaggctgggcttaagaaggacgatggagaaggacggggaacgccagaagctgaagtcaattaa

SEQ ID NO: 10 - DNA, 1711 bp, Neospora caninum
aattcggcacgagtttttcgtcatttcccttgtaagctgtgtcaagccgtttttagaaccaataaagcctatctctgcgtaggcattcttcttttttgcagtagaggcttctatttcactgaaccattgtgccttcgctaccggacgggtgcgtagtttgagtcgtaaccggggctcaaccgtggcagtccgctgttttgcggatacgctgtcattgtggtcctttcgttcattttcgtgatttccttcccttgtagtgacttcctcggcactctgcctttagttaacgtttaaaattcagctttgttgtcgcgactgcattccaatagtccaggaagagatttgtgcacgtggcggaccgagccagcgacctcgtggaggcttgacgtgacgtgcagcagcaagaggcaagagaaggtgcgtgcgccgcccacagccaaggtcaacttacggtagcataataggactctttttgtgctgttgagcgattccgaaacaactcgaaaagaaaggacttcgtgggaggccgtaactgtcgtcgtcctggtgtgttttccaaaccactgctcaactacatttttaccgcttcaccaccatctgttgcgctccgaggtagtgcagaggcacagtctccccgtgcaactatatttgaaggaaacatggatcctaaagtggagagtcaaacaaatgtgccatctggcgcagaggcagagcagcccaaggcaggagaggcacaagcaactgtggagaacggtaatacttcagctccggatgctcaggtgaagtcccaagcgtcctccgaagatgtggtagcgcagtcgtcagaagacttcagcggaaagcttcaggccaactcaggcattgtgagcttcggagactctgctgctggaagtggtgcgttcaacagtatggacgtgcagaactttctccagcgttacgcaacgagcaagatgtttggagttccgccgcatttcttccaaagcagagaaagcctccgagtctggggagctgaccacctcaccgatcccatggtgcagccttacgagaaagacgatcagaacctacccaatccctttcatgtttcgctacctgggtactctccgtctctctgcaagtacgttctgaccaagggcgagaagcctccccgcgatcccctcctcggacctgagattaccatttacccgcctacgtggattccgcactgggaacccgatcccaatttcaagccacaggcttacaatttcaactgggaggagaacggcacatttcagatggaacggttgccgtacgcgaaagcggtcttcgatccagcagacggctcagcacacggcatgtacaagcaagcctacccttacacagcgtatccatacggtgttccgcgcgtctagatagcataaacattgttttcctcttgggataaaagcacaggcaaaacaagggatcgttcctcttagtcaacgactgctgaacagcagtcagtcagttcagggcgtggccctgacgggttcatcagcccatttttttggtcgagtcactgtttgttccggggatctggctgtggcaccgaaggcaatcttgccttgctgcctataaaaattcctcattctgtttgtacgcttactaagcttcctggcctcgtcgtttggctgtggtccatcctctacaaacttatctccatcctcaacaaggccataaaaaacctgttttattc

SEQ ID NO: 11 - DNA, 2434 bp, Neospora caninum
taagctgtgtcaagccgtttttagaaccaataaagcctatctctgcgtaggcattcttcttttttgcagtagaggcttctatttcactgaaccattgtgccttcgctaccggacgggtgcgtagtttgagtcgtaaccggggctcaaccgtggcagtccgctgttttgcggatacgctgtcattgtggtcctttcgttcattttcgtgatttccttcccttgtagtgacttcctcggcactctgcctttagttaacgtttaaaattcagctttgttgtcgcgactgcattccaatagtccaggaagagatttgtgcacgtggcggaccgagccagcgacctcgtggaggcttgacgtgacgtgcagcagcaagaggcaagagaaggtgcgtgcgccgcccacagccaaggtcaacttacggtagcataataggactctttttgtgctgttgagcgattccgaaacaactcgaaaagaaaggacttcgtgggaggccgtaactgtcgtcgtcctggtgtgttttccaaaccactgctcaactacatttttaccgcttcaccaccatctgttgcgctccgaggtagtgcagaggcacagtctccccgtgcaactatatttgaaggaaacatggatcctaaagtggagagtcaaacaaatgtgccatctggcgcagaggcagagcagcccaaggcaggagaggcacaagcaactgtggagaacggtaatacttcagctccggatgctcaggtgaagtcccaagcgtcctccgaagatgtggtagcgcagtcgtcagaagacttcagcggaaagcttcaggccaactcaggcattgtgagcttcggagactctgctgctggaagtggtgcgttcaacagtatggacgtgcagaactttctccagcgttacgcaacgagcaagatgtttggagttccgccgcatttcttccaaagcagagaaagcctccgagtctggggagctgaccacctcaccgatcccatggtgcagccttacgagaaagacgatcagagtaaggtcatagcacaccgtattctggacagaatcagcgacgagtacgatagtcttgctgaacgatgggagtaggatttttcgtcctccttgcatcgacggagatatgaccctctgacggacgcagcagtaccaccattatccactgtcatgtacttcactgtatgtgacctgtctacatcaagtcttccatatggatgtttcgatcgtatctagcaaggatgtagtatgtttgcttaaccgcaagctagggggggaggggggatgtcgctcgtctgtttgaatagcaagtgatggttatgtagatgtctctttgctatgctgtttttacagacctacccaatccctttcatgtttcgctacctgggtactctccgtctctctgcaagtacgttctgaccaagggcgagaagcctccccgcgatcccctcctcggacctgagattaccatttacccgcctacgtggattccgcactgggaacccgatcccaatttcaagccacaggcttacaatttcaactgtaagttggcgtgcaacgcagccgatcgtgtggaggcttgatttttctgcggaaagagcgtgcatgcgatacagcactttcctaatttttattgtgaacgccacatgccgaggtgcgctcctccgtatgtaaaagtcccgggtcagcttgaggtagtcgatatcagtgagacacacatacaaagcttagggactctcctgttttctgttttccacagtctttcattcaaatactttcatacaaatactgaaaacccctcggtaaactcatagaaaaaccaaagtatttttgcctgtaaccagcgtctctactagctgctggttttgtttcaccatcgtactagatgacggtatatccacgcagaactatggtttaatatctggcgttcccctgttttcgattatgtgtgtgaaattgctcaggggaggagaacggcacatttcagatggaacggttgccgtacgcgaaagcggtcttcgatccagcagacggctcagcacacggcatgtacaagcaagcctacccttacacagcgtatccatacggtgttccgcgcgtctagatagcataaacattgttttcctcttgggataaaagcacaggcaaaacaagggatcgttcctcttagtcaacgactgctgaacagcagtcagtcagttcagggcgtggccctgacgggttcatcagcccatttttttggtcgagtcactgtttgttccggggatctggctgtggcaccgaaggcaatcttgccttgctgcctataaaaattcctcattctgtttgtacgcttactaagcttcctggcctcgtcgtttggctgtggtccatcctctacaaacttatctccatcctcaacaaggccataaaaaacctgttttattca

SEQ ID NO: 12 - DNA, 1387 bp, Neospora caninum
  modified_base (1276)..(1277): a, c, g, t, other or unknown
cgagcacccacaagtaactgtgttgactatttactgctgtttttgcgtagcaccacacgatgttcacggggaaacgttggatacttgttgttgccgttggcgccctggtcggcgcctcggtaaaggcagccgatttttctggcaggggaaccgtcaatggacagccggttggcagcggttattccggatatccccgtggcgatgatgttaggtaggttaccacaacttgctgcgaacccaagggttaaagggtagagctggctagattttccaacactgtatcatgtacctccgtctgtttcatcgggcagtagtagcatgggagtgctcgtcacaagccgttgggggcaaggtttctgttgtcttgccatgcgtgtatcgcccgctcctggttcatgcttatatgcgatctagtgccccacgcgcgatgctcaatgcaattgccttttgcagagaatcaatggctgcacccgaagatctgccaggcgagaggcaaccggagacacccacggcggaagctgtaaaacaggcagcggcaaaagcttatcgattactcaagcagtttactgcgaaggtcggacaggaaactgagaacgcctactaccacgtgaagaaagcgacaatgaaaggctttgacgttgcaaaagaccagtcgtataagggctacttggccgtcaggaaagccacagctaagggcctgcagagcgctggcaagagccttgagcttaaagagtcggcaccgacaggcactacgactgcggcgccgactgaaaaagtgccccccagtggcccgtgatcaggtgaagttcaacgtactcgtaaggagcaaaatgacgtgcagcaaaccgcagagatgttggctgaggaaattcttgaggctgggcttaagaaggacgatggagaaggacggggaacgccagaagctgaagtcaattaagaaaatcactaaacgtcaagttctttatgactgctgtacaccaccacccccctggactgcttaagacagctaacaagcgttggatttcaatatcctacttaaggtatgtggggcggatgtcgtgtcacggtgtgtatggcgttaaaaaacggcacacggcattaaatgcagtgcaagtatgaattgtgcgcaggatgacaacatctgttgcaaacagctcttgggggcgaacgagaatgagaccgttgcattcgcgtacgtgcatacgatggcccattttcgggtgccaatagttgtgtgtgacatttttcggatgtcctgggctttgtgtgcgtgcgnnggctgcgaagagnattagatttatttcttgcgantgcnannnntantttgttgcatccgttatggtcatgaaaaaattgctaacgacacacataaacgatggagcaaatt

SEQ ID NO: 13 - protein, 185 aa, Toxoplasma gondii
MFAVKHCLLVVAVGALVNVSVRAAEFSGVVNQGPVDVPFSGKPLDERAVGGKGEHTPPLPDERQQEPEEPVSQRASRVAEQLFRKFLKFAENVGHHSEKAFKKAKVVAEKGFTAAKTHTVRGFKVAKEAAGRGMVTVGKKLANVESDRSTTTTQAPDSPNGLAETEVPVEPQQRAAHVPVPDFSQ

SEQ ID NO: 14 - DNA, 31 bp, Artificial Sequence
  Description of Artificial Sequence PCR primer
acgtatggatccggctttgtctacgatgaac

SEQ ID NO: 15 - DNA, 30 bp, Artificial Sequence
  Description of Artificial Sequence PCR primer
acgcatgaattctgtttctgagttcccgct

SEQ ID NO: 16 - DNA, 17 bp, Artificial Sequence
  Description of Artificial Sequence PCR primer
gtaaaacgacggccagt

SEQ ID NO: 17 - DNA, 15 bp, Artificial Sequence
  Description of Artificial Sequence PCR primer
gccgctctagaacta

SEQ ID NO: 18 - DNA, 17 bp, Artificial Sequence
  Description of Artificial Sequence PCR primer
cgagcacccacaagtaa

SEQ ID NO: 19 - DNA, 18 bp, Artificial Sequence
  Description of Artificial Sequence PCR primer
gaccataacggatgcaac

SEQ ID NO: 20 - DNA, 18 bp, Artificial Sequence
  Description of Artificial Sequence PCR primer
cagcggttattccggata

SEQ ID NO: 21 - DNA, 20 bp, Artificial Sequence
  Description of Artificial Sequence PCR primer
gcctcaagaatttcctcagc

SEQ ID NO: 22 - DNA, 20 bp, Artificial Sequence
  Description of Artificial Sequence PCR primer
ggtaggttaccacaacttgc

SEQ ID NO: 23 - DNA, 18 bp, Artificial Sequence
  Description of Artificial Sequence PCR primer
gcaattgcattgagcatc

SEQ ID NO: 24 - DNA, 31 bp, Artificial Sequence
  Description of Artificial Sequence PCR primer
acggatggatccgttcacggggaaacgttgg

SEQ ID NO: 25 - DNA, 30 bp, Artificial Sequence
  Description of Artificial Sequence PCR primer
acgtcagaattctaacgccatacacaccgt

SEQ ID NO: 26 - DNA, 21 bp, Artificial Sequence
  Description of Artificial Sequence PCR primer
gaggtatatattaatgtatcg

SEQ ID NO: 27 - DNA, 39 bp, Artificial Sequence
  Description of Artificial Sequence PCR primer
cgtacgtctagagccaccatgttcacggggaaacgttgg

SEQ ID NO: 28 - DNA, 30 bp, Artificial Sequence
  Description of Artificial Sequence PCR primer
acgtcaggatccgcacgcacacaaagccca

SEQ ID NO: 29 - DNA, 20 bp, Artificial Sequence
  Description of Artificial Sequence PCR primer
gctgacagactaacagactg

SEQ ID NO: 30 - DNA, 18 bp, Artificial Sequence
  Description of Artificial Sequence PCR primer
aactagaaggcacagcag

SEQ ID NO: 31 - DNA, 39 bp, Artificial Sequence
  Description of Artificial Sequence PCR primer
cgtacgtctagagccaccatggtcggcgccgcagtcgta

SEQ ID NO: 32 - DNA, 30 bp, Artificial Sequence
  Description of Artificial Sequence PCR primer
acgtcaggatccttcacggggaaacgttgg

SEQ ID NO: 33 - protein, 13 aa, Artificial Sequence
  Description of Artificial Sequence Signal peptide
WILVVAVGALVGA

SEQ ID NO: 34 - DNA, 18 bp, Artificial Sequence
  Description of Artificial Sequence PCR primer
accgtggcagtccgctgt

SEQ ID NO: 35 - DNA, 18 bp, Artificial Sequence
  Description of Artificial Sequence PCR primer
tgggctgatgaccccgtc

SEQ ID NO: 36 - DNA, 18 bp, Artificial Sequence
  Description of Artificial Sequence PCR primer
ccaaggcaggagaggcac

SEQ ID NO: 37 - DNA, 16 bp, Artificial Sequence
  Description of Artificial Sequence PCR primer
accactgctcaactac

SEQ ID NO: 38 - DNA, 16 bp, Artificial Sequence
  Description of Artificial Sequence PCR primer
gcgcgtctagatagca

SEQ ID NO: 39 - DNA, 16 bp, Artificial Sequence
  Description of Artificial Sequence PCR primer
gcgcgtctagatagca

SEQ ID NO: 40 - DNA, 16 bp, Artificial Sequence
  Description of Artificial Sequence PCR primer
agcctatctctgcgta

SEQ ID NO: 41 - DNA, 19 bp, Artificial Sequence
  Description of Artificial Sequence PCR primer
agctgaccacctcaccgat

SEQ ID NO: 42 - DNA, 19 bp, Artificial Sequence
  Description of Artificial Sequence PCR primer
tgaagtcccaagcgtcctc

SEQ ID NO: 43 - DNA, 19 bp, Artificial Sequence
  Description of Artificial Sequence PCR primer
actctccgtctctctctgc

SEQ ID NO: 44 - DNA, 18 bp, Artificial Sequence
  Description of Artificial Sequence PCR primer
ccacgccctgaactgact

SEQ ID NO: 45 - DNA, 17 bp, Artificial Sequence
  Description of Artificial Sequence PCR primer
gccttgttgaggatgga

SEQ ID NO: 46 - DNA, 15 bp, Artificial Sequence
  Description of Artificial Sequence PCR primer
tgctggatcgaagac

SEQ ID NO: 47 - DNA, 17 bp, Artificial Sequence
  Description of Artificial Sequence PCR primer
aggcgggtaaatggtaa

SEQ ID NO: 48 - DNA, 31 bp, Artificial Sequence
  Description of Artificial Sequence PCR primer
acgcatggatccggatcctaaagtggagagt

SEQ ID NO: 49 - DNA, 30 bp, Artificial Sequence
  Description of Artificial Sequence PCR primer
acgtatgaattcccaagaggaaaacaatgt

SEQ ID NO: 50 - DNA, 21 bp, Artificial Sequence
  Description of Artificial Sequence PCR primer
gaggtatatattaatgtatcg

SEQ ID NO: 51 - DNA, 34 bp, Artificial Sequence
  Description of Artificial Sequence PCR primer
acgcatgaattctatggatcctaaagtggagagt

SEQ ID NO: 52 - DNA, 30 bp, Artificial Sequence
  Description of Artificial Sequence PCR primer
catgacctcgaggacgcgcggaacaccgta

SEQ ID NO: 53 - DNA, 21 bp, Artificial Sequence
  Description of Artificial Sequence PCR primer
ttaatacgactcactataggg

SEQ ID NO: 54 - DNA, 18 bp, Artificial Sequence
  Description of Artificial Sequence PCR primer
gctagttattgctcagcg

SEQ ID NO: 55 - DNA, 31 bp, Artificial Sequence
  Description of Artificial Sequence PCR primer
acgtatggatccgttttgtcaggtgttcttg

SEQ ID NO: 56 - DNA, 31 bp, Artificial Sequence
  Description of Artificial Sequence PCR primer
acgtatggatccgaacaagcccgggccgttt

SEQ ID NO: 57 - DNA, 32 bp, Artificial Sequence
  Description of Artificial Sequence PCR primer
acgtataagctttgccttcttgcgggccgcga

SEQ ID NO: 58 - DNA, 21 bp, Artificial Sequence
  Description of Artificial Sequence PCR primer
atatactactccctgtgagtt

SEQ ID NO: 59 - DNA, 21 bp, Artificial Sequence
  Description of Artificial Sequence PCR primer
gtaatctgaaagcgaatagag

SEQ ID NO: 60 - protein, 4 aa, Artificial Sequence
  Description of Artificial Sequence Synthetic peptide motif
AYPY